(19) United States
(12) Patent Application Publication
(10) Pub. No.: US 2023/0227477 A1
(43) Pub. Date: Jul. 20, 2023

(54) COMPOUNDS FOR SENSING REACTIVE OXYGEN SPECIES AND METHODS FOR USING THE SAME

(71) Applicant: WAKE FOREST UNIVERSITY HEALTH SCIENCES, Winston-Salem, NC (US)

(72) Inventors: John C. Lukesh, III, Clemmons, NC (US); Katherine Virginia Ambrose, Winston-Salem, NC (US)

(21) Appl. No.: 18/002,535

(22) PCT Filed: Jun. 24, 2021
(86) PCT No.: PCT/US2021/038923

§ 371 Date: Dec. 20, 2022

Related U.S. Application Data
(60) Provisional application No. 62/705,404, filed on Jun. 25, 2020.

(51) Int. Cl. C07F 5/02

(52) U.S. Cl. CPC C07F 5/02

(57) ABSTRACT

Provided herein according to some embodiments of the invention are aryl boronate and/or aryl diaminoboryl compounds. Also provided are methods of detecting the presence of hydrogen peroxide (H₂O₂) and/or other reactive oxygen species (ROS) in a cell and/or tissue by contacting the cell and/or tissue with aryl boronate and/or aryl diaminoboryl compounds. Also provided according to embodiments of the invention are methods of producing persulfides in the presence of hydrogen peroxide (H₂O₂) and/or other reactive oxygen species (ROS) in a cell and/or tissue by contacting the cell and/or tissue with aryl boronate and/or aryl diaminoboryl compounds.

### STATEMENT OF PRIORITY

This application claims the benefit of U.S. Provisional Application Ser. No. 62/705,404, filed Jun. 25, 2020, the entire contents of which are incorporated by reference herein.

### FIELD

The present invention relates to compounds for sensing reactive oxygen species such as hydrogen peroxide. The present invention further relates to persulfide precursor compounds and methods of forming persulfide compounds.

### BACKGROUND

Hydrogen sulfide (H₂S) has recently been implicated as a key signaling molecule within mammalian systems, mediating numerous physiological processes related to inflammation, the brain and central nervous system, and the cardiovascular system. See, e.g., X. Cao, et al., Antioxid. Redox Signal. 2019, 31, 1-38; H. Kimura, Amino Acids 2011, 41, 113-121; and M. R. Filipovic, et al., Chem. Rev. 2018, 118, 1253-1337. In fact, along with nitric oxide (NO) and carbon monoxide (CO), H₂S has now established itself as an enduring member of the gasotransmitter family, a well-known class of gaseous, cell-permeable signaling molecules. See, e.g., R. Wang, Proc. Natl. Acad. Sci. 2012, 109, 8801-8802; A. Papapetropoulos, et al., Br. J. Pharmacol. 2015, 172, 1395-1396; and G. Cirino, et al., Br. J. Pharmacol. 2017, 174, 4021-4031.

Since the early 2000s there has been an exponential increase in the number of studies that have been published related to H₂S and its physiological significance. However, with protein persulfidation (Cys-SH to Cys-SSH) believed to be a primary mechanism by which H₂S relays its signaling, many of the physiological effects that were once attributed to may in fact be mediated by other reactive sulfur species (RSS), such as persulfides (RSSH) and polysulfides. See, e.g., T. Ida, et al., Proc. Natl. Acad. Sci. USA 2014, 111, 7606-7611; K. Ono, et al., Free Radic. Biol. Med. 2014, 77, 82-94; C. Yang, et al., Antioxid. Redox Signal. 2019; and N. Lau, et al., Curr. Opin. Chem. Biol. 2019, 49, 1-8.

Like hydrogen sulfide, persulfides also display enhanced nucleophilicity and are more potent reductants than their thiol counterparts within a biological setting (e.g., glutathione). See, e.g., C.-M. Park, et al., Mol. Biosyst. 2015, 11, 1775-1785 and B. Yu, et al., Antioxid. Redox Signal. 2020. This is mostly attributed to the suppressed pKₐ of RSSH which results in the more reactive RSS⁻ being its dominant form at neutral pH. See, e.g., E. Cuevasanta, et al., J. Biol. Chem. 2015, 290, 26866-26880. However, in addition to high nucleophilicity, persulfides also possess sufficient electrophilic character as well. See, e.g., W. Chen, et al., Chem. Sci. 2013, 4, 2892-2896 and W. Chen, et al., Angew. Chem. Int. Ed. 2015, 54, 13961-13965. Consequently, via thiol-persulfide exchange reactions, persulfides have the unique ability to promote the direct persulfidation of protein thiols while also serving as valuable H₂S donors themselves.

Since H₂S and RSSH enjoy chemical advantages over their thiol relatives, it likely ensures the biological importance and participation of both of these species in not only redox signaling, but in providing cellular protection as well. In earlier attempts to assess the natural biological functions of reactive sulfur species, inorganic salts, such as sodium sulfide (Na₂S) and sodium hydrosulfide (NaHS), were employed as convenient precursors to H₂S. See, e.g., R. C. O. Zanardo, et al, FASEB J. 2006, 20, 2118-2120 and W. Zhao, EMBO J. 2001, 20, 6008-6016. However, given that their addition to buffered solutions results in a rapid increase in H₂S concentrations, these inorganic salts not only engendered toxicity, but they poorly mimicked the natural slow and steady enzymatic production of hydrogen sulfide.

Given the limitations of inorganic salts in biological studies, several small molecule donor compounds have since been developed that were chemically engineered to release H₂S, and other reactive sulfur species, via hydrolysis or in response to a specific biological trigger such as cellular thiols or enzymes. See, e.g., L. Li, M. Whiteman, et al, Circulation 2008, 117, 2351-2360; Y, Zhao, et al., Org. Lett. 2014, 16, 4536-4539; P. Chauhan, et al., Org. Lett. 2017, 19, 62-65; M. M. Cerda, et al., Org. Lett. 2017, 19, 2314-2317; and Y. Zheng, et al., Angew. Chem. Int. Ed. 2017, 56, 11749-11753.

Indeed, many of these donors were shown to more closely mimic the natural production of RSS, producing more favorable physiological responses. See, e.g., M. Whiteman, et al., Expert Rev. Clin. Pharmacol. 2011, 4, 13-32; Y. Zhao, et al., J. Med. Chem. 2015, 58, 7501-7511; P. De Cicco, et al., Pharmacol. Res. 2016, 114, 67-73; and D. Gerő, et al., Pharmacol. Res. 2016, 113, 186-198. Of note, certain donors were shown to selectively release H₂S precursors in response to oxidative stress. See, e.g., Y. Zhao, et al., Angew. Chem. Int. Ed. 2016, 55, 14638-14642; C. R. Powell, et al., Angew. Chem. Int. Ed. 2018, 57, 6324-6328; P. Chauhan, et al., Org. Lett. 2018, 20, 3766-3770; P. Bora, et al., Org. Lett. 2018, 20, 7916-7920; and K. Dillon, et al., Synlett 2019, 30, 525-531. An advantage of these donors is not only their potential to further elucidate the physiological effects of H₂S (or other RSS) on systems which are under oxidative stress, but they may better gauge the therapeutic potential and ability of RSS to combat oxidative stress-related diseases.

Aryl boronate esters are known as selective, bioorthogonal triggers for hydrogen peroxide (H₂O₂)-sensing. See, e.g., A. Lippert, et al., Acc. Chem. Res. 2011, 44, 793-804; B. J. Bezner, et al., Anal. Chem. 2020, 92, 309-326; H. Ye, et al., Biomacromolecules 2019, 20, 2441-2463; C. Chung, et al., Chem. Commun. 2011, 47, 9618-9620; and L. Yi, et al., Chem.-Eur. J. 2015, 21, 15167-15172. Many of these studies have highlighted the selective release of payload, via a 1,6-elimination, from the intermediate phenol that forms as a result of boronate ester oxidation. This has resulted in the development of reaction-based fluorescent probes and prodrugs—including several RSS donors—that are selectively responsive towards H₂O₂. However, given the highly electrophilic 1,4-quinonemethide that ultimately forms via this mechanistic pathway, the physiological application of these molecules is often limited. See, e.g., T. J. Monks, et al., Toxicol. Appl. Pharmacol. 1992, 112, 2-16 and T. Monks, et al., Curr. Drug Metab. 2002, 3, 425-438.

New hydrogen peroxide-sensing motifs that would provide selective and efficient release of the desired payload without the formation of deleterious by-products would be beneficial.

### SUMMARY

Provided herein according to some embodiments of the invention is a compound of Formula I:

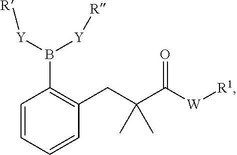

wherein:

R¹ is H, halo, alkyl, heteroalkyl, aryl, or heteroaryl, wherein the alkyl, heteroalkyl, aryl, or heteroaryl is optionally substituted, and/or R¹ comprises at least one of a prodrug moiety and a reporter moiety;

each Y is independently NRₐ or O;

W is NR_{b}, S, or O;

Rₐ and R_{b} are each independently H or alkyl, optionally substituted; and

R′ and R″ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, —OH, alkoxy, carbonyl and/or carboxy groups;

or a pharmaceutically acceptable salt thereof.

In some embodiments of the invention, the compound of Formula I is a compound of Formula Ia:

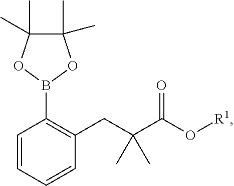

wherein R¹ is as defined above, or a pharmaceutically acceptable salt thereof.

In some embodiments of the invention, R¹ comprises a reporter moiety. In some embodiments, the reporter moiety generates a fluorescent compound when the compound of Formula I reacts with hydrogen peroxide (H₂O₂) and/or other reactive oxygen species (ROS).

Further provided according to embodiments of the invention is a compound of Formula II:

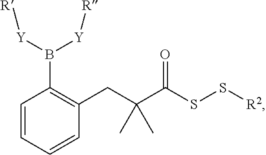

wherein:

R² is H, alkyl, heteroalkyl, aryl, or heteroaryl, wherein the alkyl, heteroalkyl, aryl, or heteroaryl is optionally substituted, and/or R² comprises at least one of a reporter moiety and a prodrug moiety,

each Y is independently NR_{c} or O;

R_{c} is H or alkyl, optionally substituted;

R′ and R″ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, —OH, alkoxy, carbonyl and/or carboxy groups; and

or a pharmaceutically acceptable salt thereof.

In some embodiments of the invention, the compound of Formula II is a compound of Formula IIa:

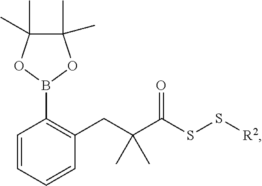

wherein R² is as defined above,

or a pharmaceutically acceptable salt thereof.

Also provided according to embodiments of the invention is a method of detecting the presence of hydrogen peroxide (H₂O₂) and/or other reactive oxygen species (ROS) in a cell and/or tissue comprising contacting the cell and/or tissue with a compound of Formula I. In some embodiments, a lactone is produced upon oxidation of the boronate or diaminoboryl group of a compound of Formula I by the hydrogen peroxide (H₂O₂) and/or other reactive oxygen species (ROS). As such, in some embodiments, detecting the presence of hydrogen peroxide (H₂O₂) and/or other reactive oxygen species (ROS) includes detecting the presence of the formed lactone. In some embodiments, R¹ of the compound of Formula I comprises a reporter moiety and the reporter moiety produces a fluorescent compound upon reaction of the compound of Formula I with hydrogen peroxide (H₂O₂) and/or other reactive oxygen species (ROS). Therefore, in some embodiments, methods of detecting hydrogen peroxide (H₂O₂) and/or other reactive oxygen species (ROS) comprise detecting the fluorescent compound produced by the reporter moiety.

Also provided according to embodiments of the invention is a method of producing a persulfide in the presence of hydrogen peroxide (H₂O₂) and/or other reactive oxygen species (ROS) in a cell and/or tissue, comprising contacting the cell and/or tissue with a compound of Formula II. The persulfide may be produced upon oxidation of the compound of Formula II with the hydrogen peroxide (H₂O₂) and/or other reactive oxygen species (ROS). In some embodiments, R² of the compound of Formula II comprises a reporter moiety and the reporter moiety produces a fluorescent compound upon reaction of the compound of Formula II with hydrogen peroxide (H₂O₂) and/or other reactive oxygen species (ROS). Therefore, in some embodiments, methods of producing the persulfide further include detecting hydrogen peroxide (H₂O₂) and/or other reactive oxygen species (ROS) and/or detecting the formation of the persulfide by detecting the fluorescent compound produced by the reporter moiety of the compound of Formula II.

In some embodiments, contacting a cell and/or tissue with a compound of Formula I and/or Formula II is carried out in vitro, and in some embodiments, contacting a cell and/or tissue with a compound of Formula I and/or Formula II is carried out in vivo.

### BRIEF DESCRIPTION OF THE DRAWINGS

FIG. 1A is a schematic illustrating a synthesis of compound 10 from tert-butyl isobutyrate. Intermediate compound 10 was accessed in three steps and with an overall yield of 62%.

FIG. 1B is a schematic illustrating the conversion of compound 10 into H₂O₂ reaction-based fluorescent probes (compounds 1a and RAH2115) and an ROS-activated persulfide donor (compound RAH393).

FIG. 2A illustrates a proposed mechanism for the detection of H₂O₂ with compound 1a. In addition to increasing the scaffold's selectivity towards H₂O₂, the gem-dimethyl group likely increases the rate of cyclization, ensuring that compound 2 does not accumulate as an unproductive intermediate along the reaction pathway.

FIG. 2B is a graph providing the time-dependent fluorescence emission (lex: 330 nm) of compound 1a (20 μM) in the presence of H₂O₂ (1000 μM). Under these conditions, the reaction of compound 1a with H₂O₂ displayed pseudo first-order kinetics and was found to have a half-life of 29.3 min. The data was plotted as the mean+/−STDEV from three independent experiments.

FIG. 3 provides data regarding the turn-on fluorescence response (lex: 330 nm) for compounds 1a, 1b, and 1c (20 μM) after a 3 h incubation period in (1) PBS buffer alone (pH 7.4, 37° C.) and (2) in the presence of H₂O₂ (1 mM). The data was plotted as the mean+/−STDEV from three independent experiments.

FIG. 4 provides the results of fluorescence experiments for compound RAH2115. Graph A of FIG. 4 shows the fluorescence response (lex: 570 nm) of compound RAH2115 (10 μM) in PBS buffer and in the presence of various oxidants, reductants, nucleophiles and salts during a 2 h incubation period at 37° C.: (1) PBS buffer alone; (2) 1 mM H₂O₂; (3) 1 mM glutathione; (4) 1 mM glutathione disulfide; (5) 1 mM NaNO₂; (6) 1 mM cysteine; (7) 1 mM lysine; (8) 1 mM serine; (9) 1 mM NaCl; (10) 1 mM NaBr; (11) 1 mM homocysteine; (12) 100 μM Na2S; (13) 1 mM sodium sulfate; (14) 1 mM sodium thiosulfate; (15) 1 mM NaOCl. The data was plotted as the mean+/−STDEV from three independent experiments. Graph B of FIG. 4 shows the change in initial rates of fluorescence intensity of compound RAH2115 (10 μM) in the presence of increasing concentrations of H₂O₂. Excellent linearity was observed between 0 and 1 mM with an R2 of 0.99. The data was plotted as the mean+/−STDEV from three independent experiments.

FIG. 5A provides a visualization of endogenous H₂O₂ in live HeLa cells with compound RAH2115 (10 μM). Imaging: ex: 561 nm, em: 587 nm. (a) and (b): In the absence of paraquat (control); (c) and (d): With the addition of paraquat (10 mM), a known reactive oxygen species (ROS) stimulator. Scale bar set to 100 μm.

FIG. 5B is a graph comparing the fluorescence intensity of cellular images in FIG. 5A. Column 1: In the absence of paraquat (control); Column 2: With the addition of paraquat (10 mM). Values are expressed as the mean+/−STDEV over three separate images.

FIG. 6A illustrates a proposed mechanism for the monitoring of persulfide release from compound 4a (20 μM) in the presence of H₂O₂ (200 μM).

FIG. 6B is a graph showing the time-dependent fluorescence emission (lex: 330 nm) of compound 4a (20 μM) in the presence and absence of H₂O₂ (200 μM). As a control, compound 4b was also assayed under the same conditions. Together, these results indicate that the observed increase in fluorescence intensity is directly proportional to persulfide release. Under these conditions, the reaction between compound 4a and H₂O₂ displayed pseudo first-order kinetics with a half-life of 60.5 min and a k_{obs} of 0.012 min-1. The data was plotted as the mean+/−STDEV from three independent experiments.

FIG. 7 illustrates the method used to test compound RAH393 as an ROS-activated persulfide donor. DNFB was used in an effort to trap the released persulfide (compound 6) as a stable disulfide (compound 7). The formation of both compound 7 and compound 3 was confirmed via LCMS.

FIG. 8 shows the viability of HeLa cells upon pre-treatment with compound RAH393 (200 μM) or compound 5 (200 μM) for 1 h followed by exposure to H₂O₂ (200 μM) for an additional 1 h. Results are expressed as the mean+/−STDEV (n=3-6 for each treatment group).

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention.

All publications, patent applications, patents, nucleotide sequences, amino acid sequences and other references mentioned herein are incorporated by reference in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

As used herein in the accompanying chemical structures, “H” refers to a hydrogen atom. “C” refers to a carbon atom. “N” refers to a nitrogen atom. “S” refers to a sulfur atom. “O” refers to an oxygen atom.

As understood in the art, the term “optionally substituted” indicates that the specified group is either unsubstituted or substituted by one or more suitable substituents. A “substituent” that is “substituted” is a group which takes the place of one or more hydrogen atoms on the parent organic molecule.

“Alkyl,” as used herein, refers to a saturated straight or branched chain, or cyclic hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-decyl, cyclopropyl, cyclobutyl, cyclopentyl, cyclohexyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, cyclopropyl, cyclobutyl, and the like. Alkyl may optionally be substituted with one or more suitable substituents, such as halo, hydroxy, carboxy, carbonyl, amino, mercapto, thiol, carboxyalkyl (e.g., carboxymethyl, carboxyethyl), and the like.

“Heteroalkyl,” as used herein, means an alkyl, as defined herein, where at least one carbon atom is replaced with a heteroatom selected from the group consisting of O, N, Si and S, wherein the nitrogen and sulfur atoms may optionally be oxidized, and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. The heteroalkyl may optionally be substituted with one or more suitable substituents, such as halo, hydroxy, carboxy, carbonyl, amino, mercapto, thiol, carboxyalkyl (e.g., carboxymethyl, carboxyethyl), and the like.

“Aryl,” as used herein, refers to a monocyclic carbocyclic ring system or a bicyclic carbocyclic fused or directly adjoining ring system having one or more aromatic rings. Examples include, but are not limited to, phenyl, indanyl, indenyl, tetrahydronaphthyl, and the like. As noted, in some embodiments, the aryl has two aromatic rings, which rings are fused or directly adjoining. Examples include, but are not limited to, biphenyl, naphthyl, azulenyl, etc. The aryl may optionally be substituted with one or more suitable substituents, such as halo, hydroxy, carboxy, carbonyl, amino, mercapto, thiol, carboxyalkyl (e.g., carboxymethyl, carboxyethyl), and the like.

“Heteroaryl,” as used herein, refers to a monovalent aromatic group having a single ring or two fused or directly adjoining rings and containing in at least one of the rings at least one heteroatom (typically 1 to 3) independently selected from nitrogen, oxygen and sulfur. Examples include, but are not limited to, pyrrole, imidazole, triazole, oxazole, furan, thiophene, triazole, pyrazole, isoxazole, isothiazole, pyridine, pyrazine, pyridazine, pyrimidine, triazine, and the like. As noted, in some embodiments, the heteroaryl has two aromatic rings, which rings are fused or directly adjoining. Examples include, but are not limited to, benzothiophene, benzofuran, indole, benzimidazole, benzothiazole, quinoline, isoquinoline, quinazoline, quinoxaline, phenyl-pyrrole, phenyl-thiophene, etc. The heteroaryl may optionally be substituted with one or more suitable substituents, such as halo, hydroxy, carboxy, carbonyl, amino, mercapto, cyano. carboxyalkyl (e,g., carboxymethyl, carboxyethyl), and the like.

The terms “halo” and “halogen,” as used herein, refer to fluoro (—F), chloro (—Cl), bromo (—Br), or iodo (—I).

The term “hydroxy,” as used herein, refers to —OH.

The term “carboxy,” as used herein, refers to —C(═O)OH.

The term “carbonyl,” as used herein, refers to —C(═O)—.

The term “amino,” as used herein, refers to —NH₂, —NHRₓ, or —NRₓR_{y}, wherein Rₓ and R_{y} are each independently alkyl, heteroalkyl, aryl, or heteroaryl, as defined herein.

The term “mercapto,” as used herein, refers to —SH.

The term “cyano,” as used herein, refers to —CN.

The term “carboxyalkyl,” as used herein, refers to —C(═O)OR, wherein R is alkyl as defined herein.

“Persulfide donor,” also referred to as “RSS,” as used herein, is a moiety or compound that produces a persulfide (RSSH). See, e.g., US 2018/0118674 to Wang et al.

Boronate or diaminoboryl-containing groups such as boronate esters, as used herein, are represented by the following general formula:

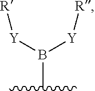

wherein each Y is independently O or NRₐ, Rₐ is H or alkyl, optionally substituted, and R′ and R″ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, —OH, alkoxy, carbonyl and/or carboxy groups. For additional groups that may be substituted on the boronate or diaminoboryl-containing groups, see US 2020/0172561 to Burn et al. In particular embodiments, the groups R′ and R″ together with the Y atoms to which they are attached form a stable cyclic boronate or di aminoboryl group.

Non-limiting examples of suitable boronate or diaminoboryl groups include, but are not limited to:

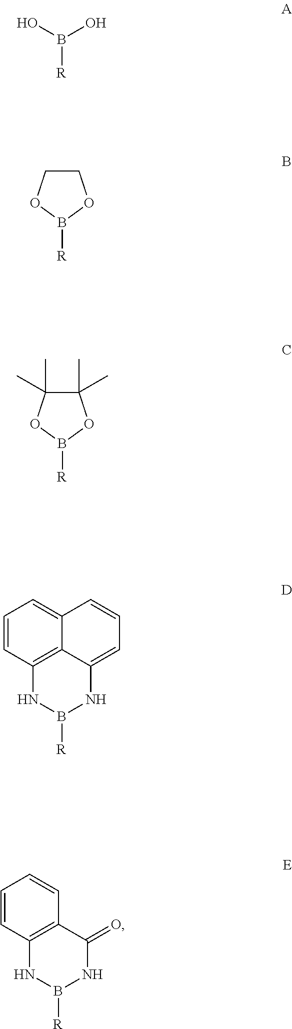

wherein R denotes attachment to the remainder of the molecule. The dimethylboronate ester (moiety C) may be easily hydrolyzed and hence, in some embodiments, the R′ and R″ together with the Y atoms to which they are attached may form moiety C, also referred to as a pinacol ester moiety.

“Reporter moiety,” as used herein, is a moiety present in a compound that is detectable (e.g., by fluorescence) upon reaction of the compound with hydrogen peroxide (H₂O₂) and/or other reactive oxygen species (ROS). In some embodiments, a reporter moiety is a portion of a compound of Formula I or II that produces a fluorescent compound upon reaction (including, e.g., cleavage and/or cyclization) of the compound of Formula I or II with hydrogen peroxide (H₂O₂) and/or other reactive oxygen species (ROS).

A “prodrug” is a drug or pharmaceutical agent that is inactive in its administered form but becomes a pharmacologically active agent by a metabolic or physico-chemical transformation. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein in their entireties. See also US 2020/0010432 to Huigens, III, et al., U.S. Pat. Nos. 5,696,126 and 6,680,299, and “Prodrug Design of Phenolic Drugs,” DOI: 10.2174/138161210791293042, herein incorporated by reference. As used herein, a “prodrug moiety” is a portion of a compound of Formula I or II, that upon reaction of the compound Formula I or II with hydrogen peroxide and/or other reactive oxygen species (ROS), is released to form an active pharmacological agent or drug.

A “pharmaceutically acceptable salt,” as used herein, is a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

Reactive oxygen species (ROS), as used herein, refer to partially reduced or excited forms of oxygen, such as, for example, peroxides (e.g., hydrogen peroxide), superoxide, hydroxyl radical, singlet oxygen, and alpha-oxygen.

Provided herein according to some embodiments of the invention is a compound of Formula I:

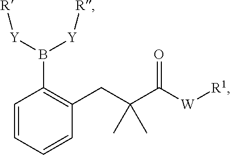

wherein:

R¹ is H, halo, alkyl, heteroalkyl, aryl, or heteroaryl, wherein the alkyl, heteroalkyl, aryl, or heteroaryl is optionally substituted, and/or R¹ comprises at least one of a prodrug moiety and a reporter moiety;

each Y is independently NRₐ or O;

W is NR_{b}, S, or O;

Rₐ and R_{b} are each independently H or alkyl, optionally substituted; and

R′ and R″ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, —OH, alkoxy, carbonyl and/or carboxy groups;

or a pharmaceutically acceptable salt thereof.

In some embodiments of the invention, Y is NH or O, W is NH or O, R′ and R″ together with the Y atoms to which they are attached form a cyclic structure containing up to 5 carbon atoms, optionally substituted by one or more alkyl (e.g., lower alkyl) and R¹ is a reporter moiety or a prodrug moiety.

Also provided herein according to some embodiments is a compound of Formula Ia:

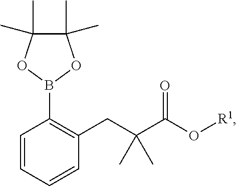

wherein R¹ is H, halo, alkyl, heteroalkyl, aryl, or heteroaryl, wherein the alkyl, heteroalkyl, aryl, or heteroaryl is optionally substituted, and/or R¹ comprises at least one of a prodrug moiety and a reporter moiety. In particular embodiments, R¹ comprises a reporter moiety and/or a prodrug moiety.

Further provided herein according to some embodiments of the invention is a compound of Formula II:

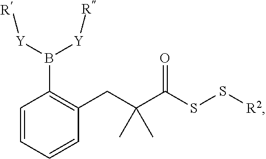

wherein:

R² is H, alkyl, heteroalkyl, aryl, or heteroaryl, wherein the alkyl, heteroalkyl, aryl, or heteroaryl is optionally substituted, and/or wherein R² comprises at least one of a reporter moiety and a prodrug moiety,

each Y is independently NR_{c} or O;

R_{c} is H or alkyl, optionally substituted; and

R′ and R″ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, —OH, alkoxy, carbonyl and/or carboxy groups;

or a pharmaceutically acceptable salt thereof.

Also provided herein according to some embodiments is a compound of Formula IIa:

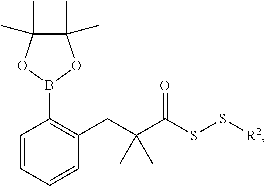

wherein R² is H, alkyl, heteroalkyl, aryl, or heteroaryl, wherein the alkyl, heteroalkyl, aryl, or heteroaryl is optionally substituted, and/or wherein R² comprises a reporter moiety.

In some embodiments, R² is —(CH₂)₂NHC(═O)CH₃.

In some embodiments, R¹ comprises a prodrug moiety and the compound of Formula I acts as a prodrug. In some embodiments, R² comprises a prodrug moiety and the compound of Formula II acts as a prodrug. When a compound of Formula I or II (e.g., a compound of Formula Ia or IIa, respectively) reacts with hydrogen peroxide (H₂O₂) and/or other reactive oxygen species (ROS), the prodrug moiety may be released to form the active drug, for example, as shown in the following scheme:

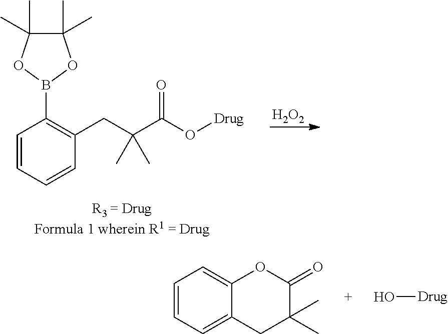

In some embodiments, the prodrug moiety is a phenolic moiety that forms a compound of Formula I wherein W is O, and the compound of Formula I is the ester derivative of the phenolic drug. Particular examples of phenolic drugs include, but are not limited to, camptothecin, SN-38, and doxorubicin. The phenolic drug R¹—OH may be released in active form upon reaction of a compound of Formula I with hydrogen peroxide (H₂O₂) and/or other reactive oxygen species (ROS). In some embodiments, the compound of Formula II may also have a prodrug moiety attached to a portion of the R² substituent via an ester linkage.

In some embodiments, a therapeutic drug may form a prodrug with the inventive scaffold using an amide linkage. For example, in some embodiments, an amine-functional drug may form the compound Formula I wherein W is NR_{b}. The amine drug may then be released (e.g., R¹—NH₂) to form the active therapeutic drug upon reaction of the compound of Formula I with hydrogen peroxide (H₂O₂) and/or other reactive oxygen species (ROS). In some embodiments, the compound of Formula II may also have a prodrug moiety attached to a portion of the R² substituent via an amide linkage.

In some embodiments of the invention, R¹ of the compound of Formula I includes a reporter moiety. In some embodiments of the invention, R² of the compound of Formula II includes a reporter moiety. In some embodiments, the reporter moiety converts to a fluorescent compound when a compound of Formula I and/or Formula II reacts with hydrogen peroxide (H₂O₂) and/or other reactive oxygen species (ROS). In some embodiments, the reporter moiety is cleavable from the compound of Formula I and/or Formula II upon a cyclization of R¹ or R², respectively. In some embodiments, the reporter moiety is fluorescent upon cleavage from the compound of Formula I and/or Formula II. Any suitable fluorescent reporter moiety may be used. However, in some embodiments, the reporter moiety in a compound of Formula I and/or II is:

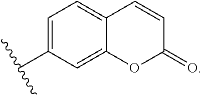 [SMILES: *c1ccc2ccc(=O)oc2c1]

In some embodiments, the reporter moiety in a compound of Formula I and/or II is:

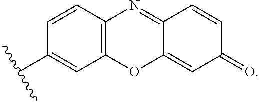 [SMILES: *c1ccc2nc3ccc(=O)cc-3oc2c1]

In some embodiments, the reporter moiety in a compound of Formula I and/or II is:

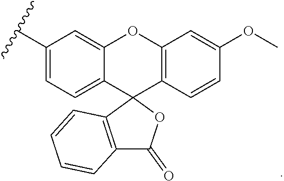 [SMILES: *c1ccc2c(c1)Oc1cc(OC)ccc1C21OC(=O)c2ccccc21]

In some embodiments, R¹ and/or R² includes:

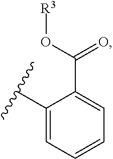

wherein R³ is the reporter moiety.

In some embodiments, a lactone is produced from a compound of Formula I and/or Formula II upon reaction with hydrogen peroxide (H₂O₂) and/or other reactive oxygen species (ROS).

In some embodiments, the cleaved persulfide from a compound of Formula II, when reacted with hydrogen peroxide (H₂O₂) or other reactive oxygen species (ROS), undergoes a cyclization to release a fluorescent reporter moiety.

Also provided according to embodiments of the invention is a method of detecting the presence of hydrogen peroxide (H₂O₂) and/or other reactive oxygen species (ROS) in a cell and/or tissue. In some embodiments, such methods include contacting the cell and/or tissue with a compound of Formula I (e.g., a compound of Formula IIa). In some embodiments, a lactone is produced upon oxidation of the boronate or diaminoboryl group of a compound of Formula I with hydrogen peroxide (H₂O₂) and/or other reactive oxygen species (ROS). As such, in some embodiments, detecting the presence of the hydrogen peroxide (H₂O₂) and/or other reactive oxygen species (ROS) includes detecting the presence of the formed lactone (e.g., via LC-MS or NMR). In some embodiments, R¹ comprises a reporter moiety and the reporter moiety produces a fluorescent compound upon reaction with the hydrogen peroxide (H₂O₂) and/or other reactive oxygen species (ROS). Therefore, in some embodiments, methods of detecting hydrogen peroxide (H₂O₂) and/or other reactive oxygen species (ROS) include detecting the fluorescent compound produced by the reporter moiety.

In some embodiments, contacting a cell and/or tissue with a compound of Formula I is carried out in vitro, and in some embodiments, contacting a cell and/or tissue with a compound of Formula I is carried out in vivo.

Also provided according to embodiments of the invention is a method of producing a persulfide compound in the presence of hydrogen peroxide (H₂O₂) and/or other reactive oxygen species (ROS) in a cell and/or tissue, comprising contacting the cell and/or tissue with a compound of Formula II (e.g., a compound of Formula IIa). The persulfide compound may be produced upon oxidation of the compound of Formula II with the hydrogen peroxide (H₂O₂) and/or other reactive oxygen species (ROS). In some embodiments, R² comprises a reporter moiety and the reporter moiety produces a fluorescent compound upon reaction with hydrogen peroxide (H₂O₂) and/or other reactive oxygen species (ROS). Therefore, in some embodiments, methods of producing the persulfide compound further include detecting the formation of the persulfide compound by detecting the fluorescent compound produced by the reporter moiety. In some embodiments, contacting a cell and/or tissue with a compound of Formula II is carried out in vitro, and in some embodiments, contacting a cell and/or tissue with a compound of Formula II is carried out in vivo.

In some embodiments, methods that include contacting a cell and/or tissue with a compound of Formula I and/or a compound of Formula II include one or more of the following steps: a) contacting a sample (e.g., a sample including cells and/or tissue) with at least one compound of the invention (a compound of Formula I and/or II); b) incubating the sample for a sufficient amount of time to allow the hydrogen peroxide or other reactive oxygen species (ROS) to react with the at least one compound of the invention to produce a fluorescent compound; c) illuminating the sample from step (b) with light of an appropriate wavelength; and d) observing the absence or presence (and optionally the concentration) of fluorescence from the sample, whereby the absence or presence of fluorescence determines the presence or absence of hydrogen peroxide and/or other reactive oxygen species (ROS) in the sample. The concentration of the fluorescence may be used to determine or estimate the concentration of hydrogen peroxide and/or other reactive oxygen species (ROS) in the sample. In some embodiments, the sample is washed prior to step (c) to remove residual, excess, or unbound compound of the invention. In some embodiments, the sample is treated with a reactive oxygen species (ROS) stimulator (e.g., paraquat) prior to or concurrent with a compound of Formula I and/or II.

The compounds of the invention may also be used to detect the presence of an enzyme (e.g., oxidase) in a sample wherein the enzyme generates hydrogen peroxide or other reactive oxygen species (ROS). The compounds of the invention may also be used to detect or monitor production of reactive oxygen species, distribution of reactive oxygen species, and metabolic activity in a cell including cell violability and proliferation.

For biological applications, the sample solution is typically an aqueous or predominantly aqueous solution that includes one or more compounds of the invention. Solutions of the compounds of the invention are prepared according to methods generally known in the art. In some embodiments, sample and/or compound solutions are aqueous or buffered aqueous solutions. Various buffers may be used that do not interfere with the fluorescent signal generated by the compound of Formula I and/or II. These buffers include PBS, Tris, MOPS, HEPES, phosphate, etc. In some embodiments, the samples are buffered to a neutral pH (e.g., pH 7.4).

Subjects, tissues, cells and proteins utilized to carry out the present invention may be of any suitable source including microbial (including gram negative and gram positive bacteria, yeast, algae, fungi, protozoa, and viral, etc.), plant (including both monocots and dicots) and animal (including mammalian, avian, reptile, and amphibian species, etc.). Mammalian subjects include both humans and other animal species treated for veterinary purposes (including but not limited to monkeys, dogs, cats, cattle, horses, sheep, rats, mice, rabbits, goats, etc.). In some embodiments, human subjects are preferred. The sample can be a biological fluid such as whole blood, plasma, serum, nasal secretions, sputum, saliva, urine, sweat, transdermal exudates, cerebrospinal fluid, or the like. Biological fluids also include tissue and cell culture medium wherein an analyte of interest has been secreted into the medium. Alternatively, the sample may be whole organs, tissue or cells from the animal. Examples of sources of such samples include muscle, eye, skin, gonads, lymph nodes, heart, brain, lung, liver, kidney, spleen, thymus, pancreas, solid tumors, macrophages, mammary glands, mesothelium, and the like. Cells include without limitation prokaryotic cells and eukaryotic cells that include primary cultures and immortalized cell lines. Eukaryotic cells include without limitation ovary cells, epithelial cells, circulating immune cells, β-cells, hepatocytes, and neurons.

In some embodiments, a fluorescent compound may be detected by use of fluorescence spectroscopy (e.g., λₑₓ=330 nm but will vary depending on the fluorophore used) and/or by the naked eye. In general, the concentration of the compound of Formula I and/or II in a sample solution is the minimum amount needed to detect the hydrogen peroxide and/or other reactive oxygen species (ROS) in a reasonable time with suitably minimal background fluorescence. In some embodiments, a compound of Formula I and/or II is present in a solution at a concentration in a range of about 10 μM to about 50 μM when detecting hydrogen peroxide and/or other reactive oxygen species (ROS) in biological cells and/or tissue.

The incubation/reaction time may vary depending upon the temperature, concentrations of cells, tissues, other reagents (e.g., a ROS stimulator) and/or the compound of Formula I and/or II. However, in some embodiments, the compound of Formula I and/or II may react with the hydrogen peroxide and/or other reactive oxygen species (ROS) in a sample for a time in a range of 10 minutes to 200 minutes. By using a specific time period for the reaction or measuring the fluorescence at 2 different times, the rate of reaction may also be determined. In some embodiments, the sample may be incubated with a compound of Formula I and/or II at a temperature in the range of about 20 to 50° C.

In some embodiments of the invention, provided are assays and kits for performing a method of the invention. Such assays and kits may include a compound of the invention (a compound of Formula I and/or Formula II) and may optionally include instructions for using the compounds of the invention, buffers, solvents and/or other known kit components.

The present invention will now be further described in the following non-limiting examples.

### EXAMPLES

### Example 1

### Synthesis of Compound 10

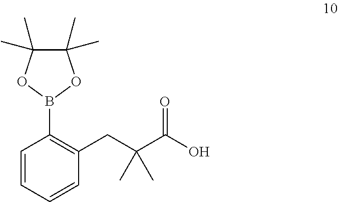 [SMILES: CC(C)(Cc1ccccc1B1OC(C)(C)C(C)(C)O1)C(=O)O]

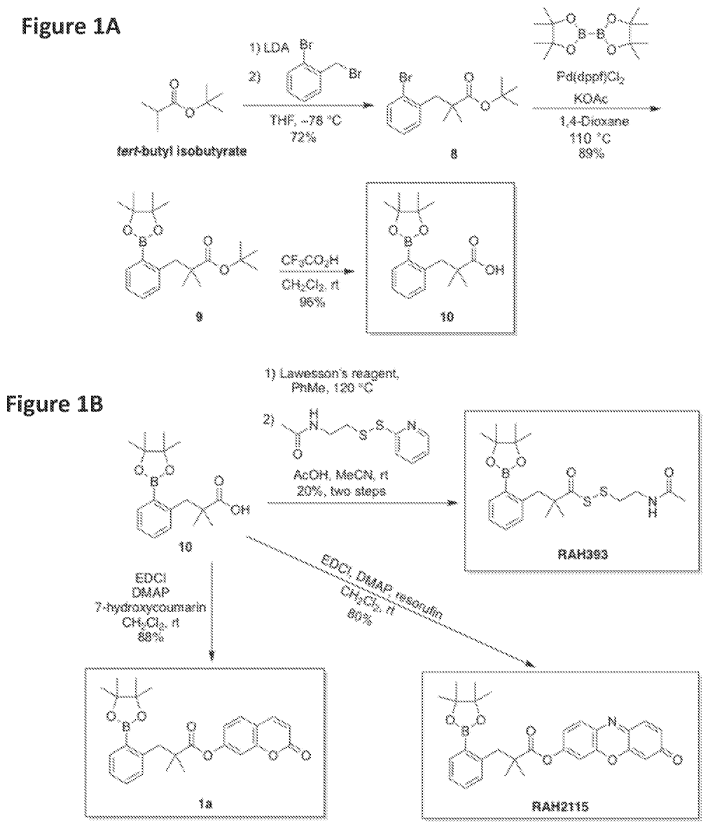

As shown in the synthetic scheme illustrated in FIG. 1A, compound 10 was first generated as it was speculated that the carboxylic acid functionality would serve as a convenient handle, enabling subsequent conjugation reactions with ease. The synthesis commenced by first treating tert-butyl isobutyrate with LDA followed by the slow addition of 2-bromobenzyl bromide which provided compound 8 in 72% yield.

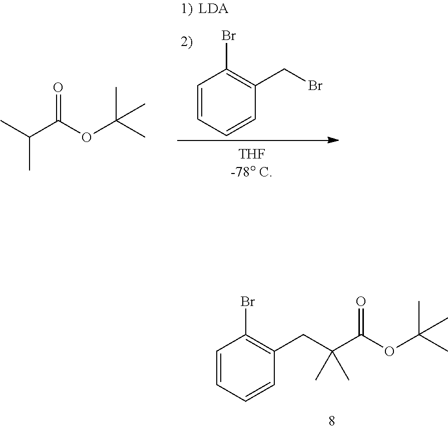

Specifically, to a solution of diisopropylamine (1.53 mL, 10.91 mmol) in anhydrous tetrahydrofuran (20 mL) at −78° C. was added n-butyllithium in hexanes (6.86 mL of a 1.6 M solution, 10.91 mmol) and the resulting mixture was stirred under nitrogen for 15 minutes. Freshly distilled tert-butyl isobutyrate (1.31 g, 9.09 mmol) dissolved in tetrahydrofuran (10 mL) was then added dropwise and stirred under nitrogen for 30 minutes. Next, 2-bromobenzyl bromide (2.50 g, 10.00 mmol) dissolved in anhydrous tetrahydrofuran (10 mL) was added dropwise, and the mixture was allowed to warm to room temperature overnight. The reaction mixture was quenched with deionized water and extracted with dichloromethane. The combined organic phase was dried over anhydrous sodium sulfate and concentrated under reduced pressure. The resulting crude oil was purified by silica gel column chromatography (100% hexanes to 15% ethyl acetate in hexanes) to obtain compound 8 as a clear oil (2.05 g, 72%). ¹H NMR (400 MHz, CDCI₃) δ=7.54 (dd, J=8.0, 1.3 Hz, 1H), 7.2.5 (dd, J=7.7, 2.1 Hz, 1H), 7.19 (td, J=7.5, 1.3 Hz, 1H), 7.05 (ddd, J=7.9, 7.1, 1.9 Hz, 1H), 3.10 (s, 2H), 1.45 (s, 9H), 1.18 (s, 6H); ¹³C NMR (101 MHz, CDCI₃) δ=176.90, 138.26, 132.97, 131.51, 127.88, 126.95, 126.15, 80.26, 44.61, 43.77, 28.00, 25.04; ESI/MS calculated for [C₁₅H₂₁BrNaO₂⁺] (M+Na⁺)⁺ requires m/z=335.06, found 335.02.

Compound 8 then underwent a Miyaura borylation reaction which furnished 9 in 89% yield.

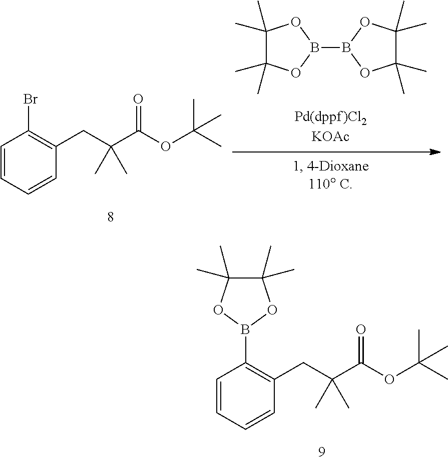

Specifically, to a solution of compound 8 (0.642 g, 2.05 mmol) in degassed 1,4-dioxane (20 mL) was added bis(pinacolato)diboron (0.628 g, 2.47 mmol), Pd(dppf)Cl₂ (154 mg, 0.21 mmol), and potassium acetate (0.607 g, 6.19 mmol) and the mixture was refluxed for 18 h under an atmosphere of nitrogen. The resulting mixture was then cooled to room temperature, concentrated under reduced pressure, and passed through a plug of silica with 15% ethyl acetate in hexanes. The solution was reduced in volume, dissolved in dichloromethane, washed with deionized water, dried over anhydrous sodium sulfate, and concentrated under reduced pressure. The crude oil was purified by column chromatography (gradient of 100% hexanes to 95:5 hexanes:ethyl acetate) to yield compound 9 as a pale-yellow oil (0.659 g, 89%). ¹H NMR (400 MHz, CDCI₃) δ=7.77 (dd, J=7.7, 1.6 Hz, 1H), 7.35-7.27 (m, 1H), 7.24-7.15 (m, 2H), 3.26 (s, 2H), 1.44 (s, 9H), 1.34 (s, 12H), 1.07 (s, 6H); ¹³C NMR (101 MHz, CDCI₃) δ=176.46, 143.83, 134.68, 129.47, 129.22, 124.39, 82.45, 78.74, 43.05, 42.16, 27.00, 23.91, 23.75; ESI/MS calculated for [C₂₁H₃₃BNaO₄⁺] (M+Na⁺) requires m/z=383.24, found 383.21.

The tert-butyl ester group was then removed from compound 9 upon treatment with trifluoroacetic acid, providing compound 10 in just three steps and with an overall yield of 62%.

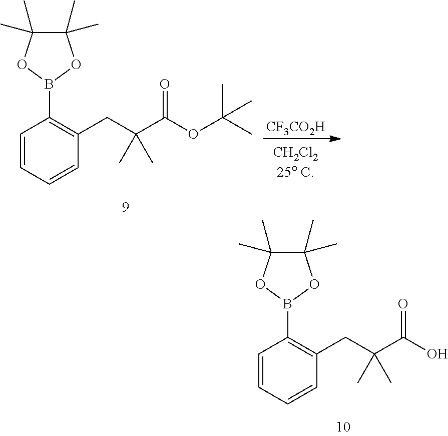 [SMILES: CC(C)(C)OC(=O)C(C)(C)Cc1ccccc1B1OC(C)(C)C(C)(C)O1 ; "CF3CO2H" ; CC(C)(Cc1ccccc1B1OC(C)(C)C(C)(C)O1)C(=O)O]

Specifically, a solution of compound 9 (0.219 g, 0.608 mmol), trifluoroacetic acid (698 μL, 9.12 mmol), and dry dichloromethane (6 mL) was stirred at room temperature under nitrogen gas until disappearance of starting material was shown by TLC. The mixture was then diluted in DCM, washed with 0.5 M HCl, dried over anhydrous sodium sulfate, and concentrated under reduced pressure. The resulting yellow oil (compound 10) required no further purification (0.177 g, 96%). ¹H NMR (400 MHz, CDCI₃) δ=7.79 (d, J=7.5 Hz, 1H), 7.33 (td, J=7.5, 1.6 Hz, 1H), 7.30-7.15 (m, 2H), 3.34 (s, 2H), 1.35 (s, 12H), 1.17 (s, 6H); ¹³C NMR (101 MHz, CDCI₃) δ=184.82, 144.14, 135.92, 130.51, 130.37, 125.72, 83.61, 43.68, 43.17, 24.94, 24.54; ESI/MS calculated for [C₁₇H₂₄BO₄⁻] (M−H⁺) requires m/z=303.18, found 303.14.

### Example 2

### Synthesis of Compound 1a

Referring to FIG. 1B and the scheme below, compound 10 was coupled with 7-hydroxycoumarin to form compound 1a.

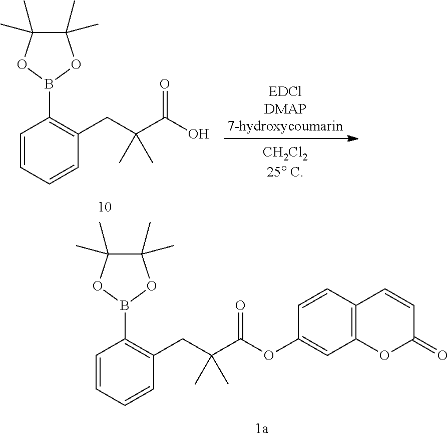 [SMILES: CC(C)(Cc1ccccc1B1OC(C)(C)C(C)(C)O1)C(=O)O ; CC(C)(Cc1ccccc1B1OC(C)(C)C(C)(C)O1)C(=O)Oc1ccc2ccc(=O)oc2c1]

Specifically, a solution of compound 10 (69 mg, 0.23 mmol) in dry dichloromethane (10 mL) was added N-ethyl-N′-(3-dimethylaminopropyl)carbodiimide hydrochloride (42 mg, 0.27 mmol), 4-dimethylaminopyridine (7 mg, 0.06 mmol) and 7-hydroxycoumarin (44 mg, 0.27 mmol) under an atmosphere of nitrogen at ambient temperature. After the mixture was allowed to stir for 18 h, the solution was washed with deionized water, dried over anhydrous sodium sulfate, and concentrated under reduced pressure. Purification was performed by silica gel chromatography (gradient of 100% hexanes to 100% ethyl acetate) to yield 1a as a white solid (90 mg, 88%). ¹H NMR (400 MHZ, CDCl₃) δ=7.84 (dd, J=7.4, 1.2 Hz, 1H), 7.69 (d, J=9.5 Hz, 1H), 7.47 (d, J=8.4 Hz, 1H), 7.37 (td, J=7.5, 1.6 Hz, 1H), 7.29-7.23 (m, 1H), 7.20 (dd, J=7.6, 0.9 Hz, 1H), 7.04 (d, J=2.2 Hz, 1H), 6.97 (dd, J=8.4, 2.2 Hz, 1H), 6.39 (d, J=9.5 Hz, 1H), 3.46 (s, 2H), 1.34 (s, 12H), 1.32 (s, 6H); ¹³C NMR (101 MHz, CDCl₃) δ=175.91, 160.47, 154.70, 153.74, 143.85, 142.93, 136.21, 130.65, 130.28, 128.43, 126.00, 118.50, 116.47, 115.94, 110.43, 83.66, 44.40, 43.58, 24.96, 24.81; ESI/MS calculated for [C₂₆H₃₀BO₆⁺] (M+H⁺) requires m/z=449.21, found 449.20.

### Example 3

### Fluorescence Studies to Assess Reactivity of Scaffold to Peroxides

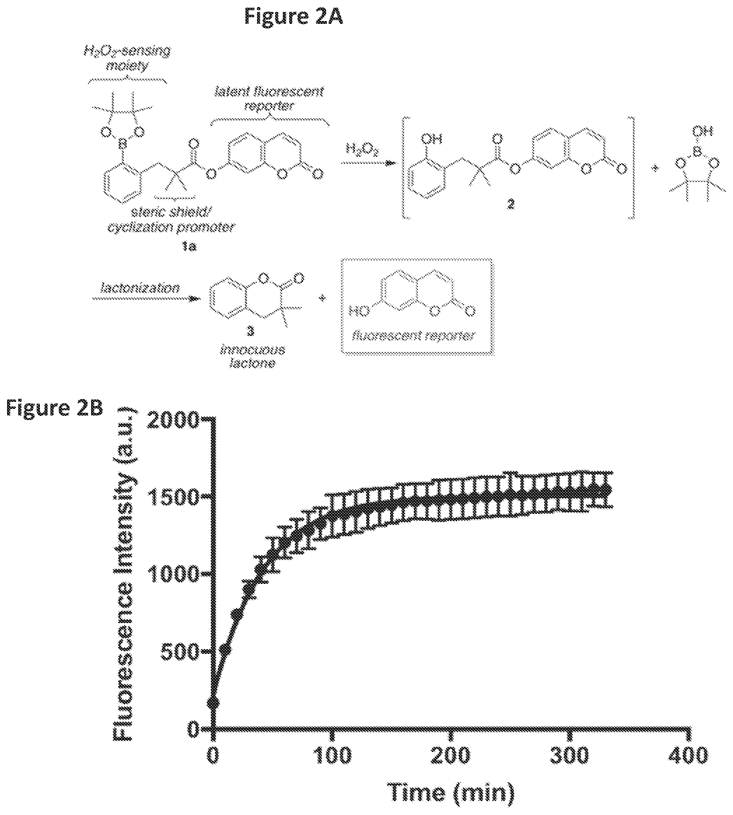

Referring to FIG. 2A, initially appending a coumarin ester to the H₂O₂-responsive scaffold of compound 1a, it was hypothesized that the release of payload, in response to H₂O₂, could be monitored via fluorescence spectroscopy while recording the resulting increase in fluorescence intensity upon excitation at 7-hydroxycoumarin's λₘₐₓ. Therefore, to initially assess the reactivity of the scaffold towards peroxides, compound 1a (20 μM) and H₂O₂ (1000 μM) was incubated in PBS buffer (pH 7.4) at 37° C. Specifically, immediately prior to use, a 5 mM stock solution of hydrogen peroxide was prepared in freshly degassed 1× PBS buffer (pH 7.4) using reagent grade H₂O₂ (30% solution). A 200 μM stock solution of probe compound 1a was prepared in 200-proof ethanol. The rate of coumarin release was measured using concentrations of 20 μM compound 1a and 1000 μM H₂O₂ by adding 30 μL of the probe stock solution, 60 μL of hydrogen peroxide stock solution and 210 μL of PBS buffer (pH 7.4) to each well of a 96 well plate. The plate was inserted into a 96-well plate reader (BioTek SYNERGY-HTX hybrid multi-mode reader, Winooski, Vt.), heated to 37° C., and agitated to ensure homogeneity. The plate was read continuously using a 360/40 nm fluorescence filter and values were recorded as the mean+/−STDEV from three experiments.

Referring to FIG. 2B, under these conditions, it was observed that the resulting fluorescence intensity increased steadily over time, displaying pseudo first-order kinetics, until it reached a maximum at roughly 1 h (t_{1/2}=29.3 min, k_{obs} of 0.024 min⁻¹), indicating complete release of the coumarin reporter. To further establish the proposed mechanistic pathway, lactone compound 3 (see FIG. 2A) was isolated and characterized as a definitive byproduct of this transformation. Intermediate compound 2 (see FIG. 2A) was not identified at any point during the reaction, however, even via LCMS, indicating that it is in fact a short-lived intermediate that is likely to cyclize immediately upon its formation. Compound 3 was formed as described below.

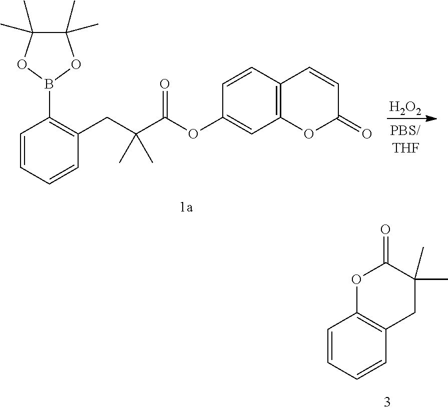

Specifically, to a solution of compound 1a (50 mg, 0.11 mmol) in tetrahydrofuran (2.74 mL) and 1× PBS buffer (2.74 mL, pH 7.4) was added a solution of 30% hydrogen peroxide (460 μL, 4.48 mmol). The solution was then heated to 37° C. and allowed to stir overnight. The mixture was then diluted with dichloromethane, washed with deionized water, dried over anhydrous sodium sulfate, and concentrated under reduced pressure. Column chromatography (gradient of 100% hexanes to 50% ethyl acetate in hexanes) afforded compound 3 as a white solid (5 mg, 26%). ¹H NMR (400 MHz, CDCl₃) δ=7.25 (td, J=7.7, 1.8 Hz, 1H), 7.16 (d, J=7.1 Hz, 1H), 7.09 (td, J=7.4, 1.2 Hz, 1H), 7.03 (d, J=8.1 Hz, 1H), 2.85 (s, 2H), 1.29 (s, 6H). ¹³C NMR (101 MHz, CDCl₃) δ=173.72, 151.51, 128.46, 128.18, 124.30, 122.07, 116.20, 38.43, 37.23, 24.70; ESI/MS calculated for [CH₁₁H₁₃O₂⁺] (M+H⁺) requires m/z=177.09, found 177.02.

### Example 4

### Synthesis of Compounds 1b and 1c

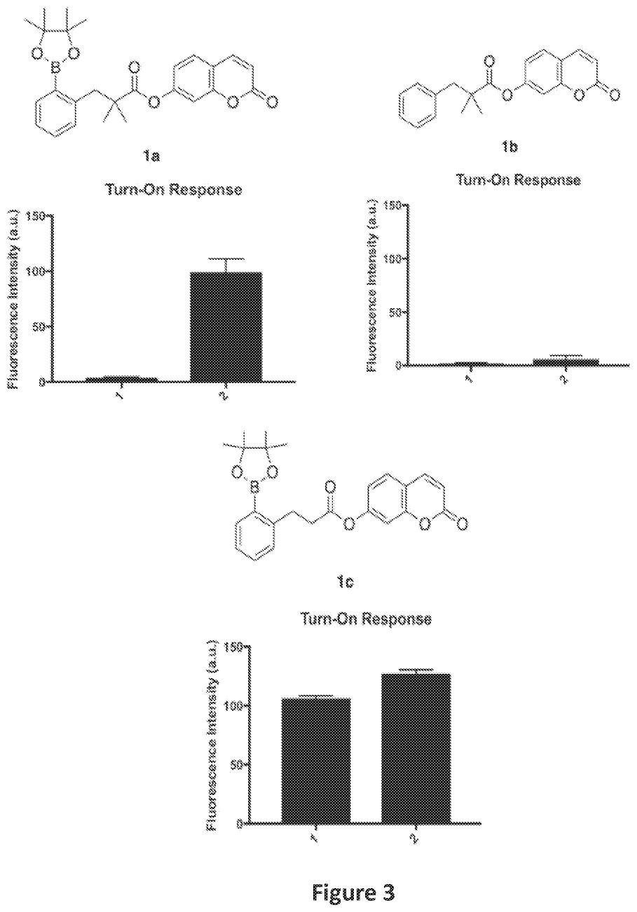

In addition to facilitating the requisite ring-closing reaction, it was further theorized that the strategic placement of the gem-dimethyl group alpha to the carbonyl would also serve as a convenient steric shield which would help reduce the nonspecific release of payload within biological systems. Referring to FIG. 3, to further examine this hypothesis, control compounds 1b (which lacked the boronate ester) and 1c (which lacked the gem-dimethyl group) were synthesized and their turn-on fluorescence was compared to that of compound 1a in both PBS buffer alone and when co-incubated with H₂O₂.

Compound 1b was formed by multi-step synthesis. First, a compound 11 was synthesized.

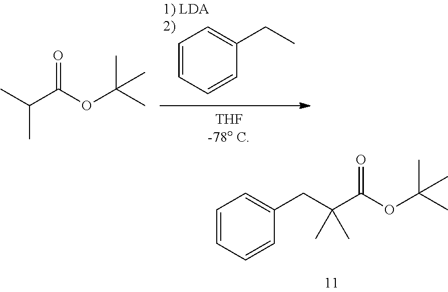

Specifically, to a solution of diisopropylamine (219 μL, 1.56 mmol) in anhydrous tetrahydrofuran (20 mL) at −78° C. was added a solution of n-butyllithium in hexanes (0.98 mL of a 1.6 M solution, 1.56 mmol) and the resulting mixture was stirred under nitrogen for 15 minutes. Freshly distilled tert-butyl isobutyrate (0.162 g, 1.42 mmol) dissolved in tetrahydrofuran (5 mL) was added dropwise and the reaction was allowed to stir under nitrogen for 30 minutes. Next, benzyl bromide (185 μL, 1.56 mmol) dissolved in anhydrous tetrahydrofuran (5 mL) was added dropwise and the reaction was allowed to warm to room temperature overnight. The reaction mixture was quenched with deionized water and extracted with dichloromethane. The combined organic extracts were dried over anhydrous sodium sulfate and concentrated under reduced pressure. The resulting crude oil was purified via column chromatography (100% hexanes to 15% ethyl acetate in hexanes) to obtain compound 11 as a clear oil (0.122 g, 37%). ¹H NMR (400 MHz, CDCl₃) δ=7.39-6.95 (m, 5H), 2.83 (s, 2H), 1.43 (s, 9H), 1.13 (s, 6H); ¹³C NMR (101 MHz, CDCl₃) δ=176.82, 138.26, 130.34, 127.85, 126.28, 80.04, 46.04, 43.85, 28.02, 25.12.

Next, compound 11 was converted to compound 12.

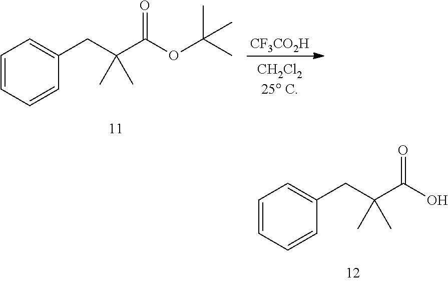 [SMILES: CC(C)(C)OC(=O)C(C)(C)Cc1ccccc1 ; "CF3CO2H" ; CC(C)(Cc1ccccc1)C(=O)O]

Specifically, to a solution of compound 11 (0.110 g, 0.47 mmol) dichloromethane (2 mL) was added trifluoroacetic acid (719 μL, 9.4 mmol) and the reaction was allowed to stir overnight at ambient temperature. The reaction mixture was diluted with dichloromethane, washed with 1M HCl, dried over anhydrous sodium sulfate, and concentrated under reduced pressure to give compound 12 as a yellow oil that required no further purification (84 mg, 100%). ¹H NMR (400 MHz, CDCl₃) δ=11.39 (s, 1H), 7.47-6.74 (m, 5H), 2.89 (s, 2H), 1.21 (s, 6H); ¹³C NMR (101 MHz, CDCl₃) δ=184.27, 137.55, 130.27, 128.07, 126.58, 45.89, 43.46, 24.69; ESI/MS calculated for [C₁₁H₁₄NaO₂⁺] (M+Na⁺) requires m/z=201.09, found 200.93.

Finally, compound 12 was reacted with 7-hydroxycoumarin to produce compound 1b.

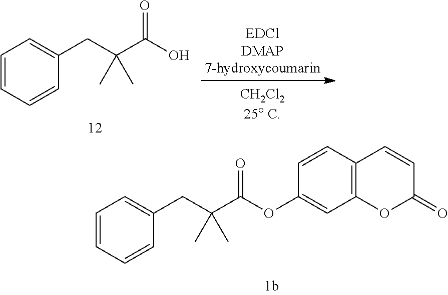 [SMILES: CC(C)(Cc1ccccc1)C(=O)O ; CC(C)(Cc1ccccc1)C(=O)Oc1ccc2ccc(=O)oc2c1]

Specifically, a solution of compound 12 (70 mg, 0.38 mmol) in dry dichloromethane (5 mL) was added N-ethyl-N′-(3-dimethylaminopropyl)carbodiimide hydrochloride (73 mg, 0.47 mmol), 4-dimethylaminopyridine (12 mg, 0.10 mmol) and 7-hydroxycoumarin (76 mg, 0.47 mmol) under an atmosphere of nitrogen at ambient temperature. The mixture was allowed to stir for 18 h and, after consumption of the starting material determined by TLC, then diluted with dichloromethane, washed with deionized water, dried over anhydrous sodium sulfate, and concentrated under reduced pressure. Purification was performed by silica gel chromatography (gradient of 100% hexanes to 100% ethyl acetate) to yield compound 1b as a white solid (0.107 g, 80%). ¹H NMR (400 MHz, CDCl₃) δ=7.68 (dd, J=9.6, 0.7 Hz, 1H), 7.47 (d, J=8.4 Hz, 1H), 7.37-7.24 (m, 3H), 7.24-7.17 (m, 2H), 6.99 (d, J=2.2 Hz, 1H), 6.94 (dd, J=8.4, 2.2 Hz, 1H), 6.39 (d, J=9.6 Hz, 1H), 3.01 (s, 2H), 1.37 (s, 6H); ¹³C NMR (101 MHz, CDCl₃) δ=175.30, 160.40, 154.70, 153.54, 142.89, 137.25, 130.22, 128.49, 128.26, 126.90, 118.43, 116.58, 116.03, 110.43, 46.38, 44.21, 25.11; ESI/MS calculated for [C₂₀H₁₉O₄⁺] (M+H⁺) requires m/z=323.13, found 323.18.

Compound 1c was produced by a two-step process. First, compound 13 was formed.

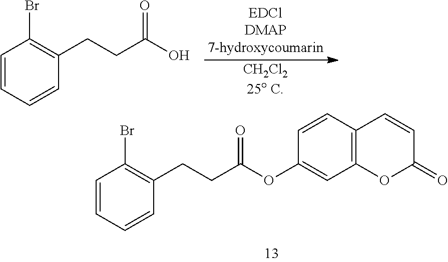 [SMILES: O=C(O)CCc1ccccc1Br ; "CH2Cl225° C." ; O=C(CCc1ccccc1Br)Oc1ccc2ccc(=O)oc2c1]

Specifically, a solution of 3-(2-bromophenyl)propionic acid (0.300 g, 1.30 mmol) in anhydrous dichloromethane (30 mL) was added N-ethyl-N′-(3-dimethylaminopropyl)carbodiimide hydrochloride (0.244 g, 1.57 mmol), 4-dimethylaminopyridine (19 mg, 0.16 mmol) and 7-hydroxycoumarin (0.255 g, 1.57 mmol). After allowing the reaction to stir overnight at ambient temperature, the reaction mixture was washed with de-ionized water, dried over anhydrous sodium sulfate, and concentrated under reduced pressure. The resultant crude oil was purified via silica gel chromatography (gradient of 100% hexanes to 5% ethyl acetate in hexanes) to yield compound 13 as a white solid (0.379 g, 78%). ¹H NMR (400 MHz, CDCl₃) δ=7.68 (d, J=9.5 Hz, 1H), 7.58 (d, J=8.0 Hz, 1H), 7.47 (d, J=8.5 Hz, 1H), 7.37-7.22 (m, 2H), 7.13 (t, J=7.6 Hz, 1H), 7.05 (s, 1H), 7.00 (d, J=8.5 Hz, 1H), 6.39 (d, J=9.6 Hz, 1H), 3.20 (t, J=7.6 Hz, 2H), 2.96 (t, J=7.6 Hz, 2H); ¹³C NMR (101 MHz, CDCl₃) δ=170.49, 160.33, 154.66, 153.10, 142.86, 139.04, 133.09, 130.68, 128.57, 128.50, 127.74, 124.39, 118.38, 116.69, 116.10, 110.42, 34.14, 31.31; ESI/MS calculated for [C₁₈H₁₄BrO⁺] (M+H⁺) requires m/z=373.01, found 372.97.

Next, compound 13 was reacted with bis(pinacolato)diboron to produce compound 1c.

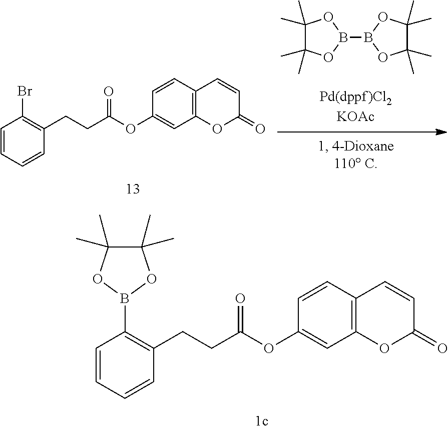

Specifically, to a solution of compound 13 (0.200 g, 0.54 mmol) in anhydrous 1,4-dioxane (20 mL) was added bis(pinacolato)diboron (0.254 g, 0.64 mmol), Pd(dppf)Cl₂ (39 mg, 0.054 mmol), and potassium acetate (0.158 g, 1.61 mmol). The resultant mixture was refluxed under nitrogen gas for 18 h. After cooling to room temperature, the solution was diluted with dichloromethane, washed with de-ionized water, concentrated under reduced pressure, and passed through a plug of silica gel with 10% ethyl acetate in hexanes. The crude solution was again concentrated under reduced pressure and further purified by silica gel chromatography (gradient of 100% hexanes to 20% ethyl acetate in hexanes) to yield 1c (64 mg, 29%). ¹H NMR (400 MHz, CDCl₃) δ=7.85 (dd, J=7.7, 1.6 Hz, 1H), 7.68 (dd, J=9.7, 0.7 Hz, 1H), 7.47 (d, J=8.4 Hz, 1H), 7.39 (td, J=7.5, 1.6 Hz, 1H), 7.26 (ddd, J=7.3, 3.8, 2.5 Hz, 2H), 7.05 (d, J=2.2 Hz, 1H), 6.98 (dd, J=8.4, 2.2 Hz, 1H), 6.39 (d, J=9.5 Hz, 1H), 3.37-3.29 (m, 2H), 2.94-2.85 (m, 2H), 1.36 (s, 12H); ¹³C NMR (101 MHz, CDCl₃) δ=171.12, 160.41, 154.70, 153.36, 146.82, 142.89, 136.61, 131.29, 129.44, 128.51, 125.95, 118.50, 116.58, 116.01, 110.48, 83.70, 37.29, 31.18, 24.93; ESI/MS calculated for [C₂₄H₂₆BO₆⁺] (M+H⁺) requires m/z=421.18, found 421.14.

### Example 5

### Turn-On Fluorescence Response of Compounds 1a, 1b, and 1c

Stock solutions of each probe (compounds 1a, 1b, 1c) at 222.2 μM were prepared in 200-proof ethanol. In addition, a 100 mM hydrogen peroxide stock solution was prepared in freshly degassed 1× PBS buffer (pH 7.4) immediately prior to use. Each reaction vial was prepared using 21.6 mL of PBS buffer, 2160 μL of compound 1a (or compound 1b or 1c) stock solution, and 240 μL of 100 mM hydrogen peroxide stock solution to give final concentrations of 20 μM of compound 1a (or compound 1b or 1c) and 1 mM of hydrogen peroxide. A control sample was prepared using 2160 μL of compound 1a (or 1b or 1c) stock solution in ethanol and diluted with 21.84 mL of freshly degassed 1× PBS buffer to give a final concentration of 20 μM of compound 1a (or 1b or 1c). The reaction and control vials were heated to 37° C. and agitated with a stir bar. At 180 min, 4 mL aliquots were transferred from reaction and control vials to individual quartz cuvettes. Fluorescence measurements (ex: 330 nm, em: 452 nm) were recorded as the mean+/−STDEV from three experiments. Referring to FIG. 3, the results for each of compounds 1a, 1b, and 1c was as follows—(1a): (1) PBS Buffer alone: 3.85+/−0.58; (2) In the presence of 1 mM H₂O₂: 97.01+/−12.97. (1b): (1) PBS Buffer alone: 0.55+/−0.27; (2) In the presence of 1 mM H₂O₂: 8.14+/−3.13. (1c): (1) PBS Buffer alone: 102.35+/−0.73; (2) In the presence of 1 mM H₂O₂: 129.54+/−0.55.

As depicted in FIG. 3, the results from this experiment indicated that compound 1a, as anticipated, was the only framework that displayed a selective turn-on fluorescence response towards H₂O₂. Conversely, compound 1b, which lacked the H₂O₂-responsive moiety, showed no turn-on response, while compound 1c, which lacked the gem-dimethyl group, was shown to be highly susceptible to hydrolysis, even in PBS buffer alone.

### Example 6

### Synthesis of Compound RAH2115

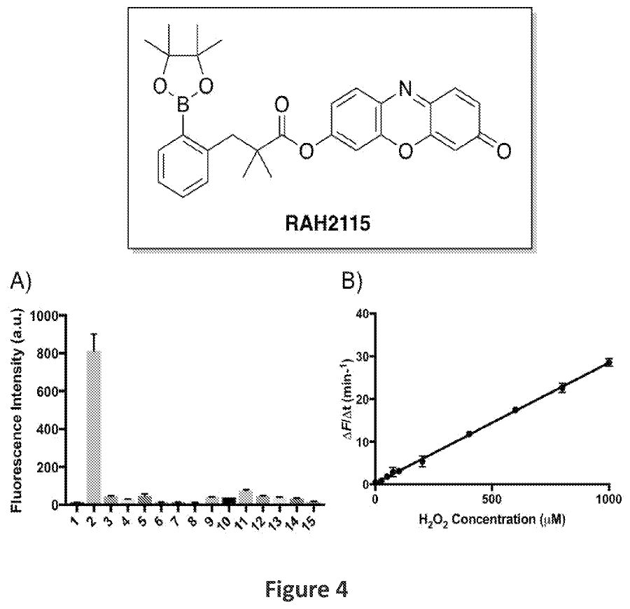

To further establish the practicality of our new hydrogen-peroxide sensing scaffold, compound RAH2115 was generated (See FIG. 1B, FIG. 4, and Scheme below).

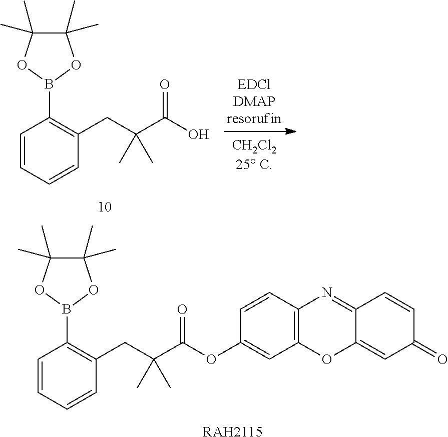

Specifically, to a solution of compound 10 (0.114 g, 0.38 mmol) in anhydrous dichloromethane (15 mL) was added resorufin (96 mg, 0.45 mmol), N-ethyl-N′-(3-dimethylaminopropyl)carbodiimide hydrochloride (70 mg, 0.45 mmol), and 4-dimethylaminopyridine (11 mg, 0.094 mmol). The mixture was allowed to stir overnight and, upon consumption of the starting material confirmed by TLC, was subsequently washed with deionized water, dried over anhydrous sodium sulfate, and concentrated under reduced pressure. The crude oil was purified via column chromatography (gradient of 100% hexanes to 100% ethyl acetate) to yield compound RAH2115 as an orange solid (0.149 g, 80%). ¹H NMR (400 MHz, CDCl₃) δ=7.85 (dd, J=7.5, 1.6 Hz, 1H), 7.79 (d, J=8.7 Hz, 1H), 7.44 (d, J=9.8 Hz, 1H), 7.38 (td, J=7.5, 1.6 Hz, 1H), 7.27 (td, J=7.4, 1.3 Hz, 1H), 7.23-7.17 (m, 1H), 7.08 (d, J=2.4 Hz, 1H), 7.04 (dd, J=8.7, 2.4 Hz, 1H), 6.87 (dd, J=9.8, 2.0 Hz, 1H), 6.34 (d, J=2.0 Hz, 1H), 3.46 (s, 2H), 1.35 (s, 12H), 1.33 (s, 6H); ¹³C NMR (101 MHz, CDCl₃) δ=186.35, 175.72, 154.10, 149.40, 148.14, 144.35, 143.78, 136.25, 135.12, 134.82, 131.11, 131.03, 130.65, 130.26, 126.02, 119.37, 109.72, 107.20, 83.68, 44.49, 43.60, 24.96, 24.81; ESI/MS calculated for [C₂₉H₃₀BNNaO₆⁺] (M+Na⁺) requires m/z=522.21, found 522.19.

### Example 7

### Fluorescence Studies with Compound RAH2115

Given the longer excitation and emission profile of resorufin, it was hypothesized that compound RAH2115 would provide a better gauge of the overall selectivity and sensitivity of the scaffold towards H₂O₂ while confirming its potential applications within a biological setting. The high selectivity the scaffold towards H₂O₂ was confirmed by thoroughly screening compound RAH2115 against various oxidants, reductants, and nucleophiles in PBS buffer (pH 7.4) and measuring the resulting turn-on fluorescence after a 2 h incubation period (See FIG. 4, Graph A).

Specifically, analyte stock solutions of hydrogen peroxide (100 mM), glutathione (100 mM), glutathione disulfide (100 mM), sodium nitrite (100 mM), L-cysteine (100 mM), L-lysine (100 mM), L-serine (100 mM), sodium chloride (100 mM), sodium bromide (100 mM), L-homocysteine (100 mM), sodium sulfide (10 mM), sodium sulfate (100 mM), sodium thiosulfate (100 mM), and sodium hypochlorite (100 mM) were prepared in freshly degassed 1× PBS buffer (pH 7.4) and used immediately prior to use. Next, 1.080 mL of a freshly prepared 111.1 μM compound RAH2115 stock solution was added to 10.80 mL of 1× PBS buffer (pH 7.4) to give a total volume of 11.88 mL. 120 μL of analyte stock solution (PBS buffer alone, hydrogen peroxide, glutathione, glutathione disulfide, sodium nitrite, L-cysteine, L-lysine, L-serine, sodium chloride, sodium bromide, L-homocysteine, sodium sulfide, sodium sulfate, sodium thiosulfate and sodium hypochlorite) was then added to give a final volume of 12 mL, and each reaction was left to stir at 37° C.

After reacting for 2 hours, fluorescence measurements (ex: 560 nm, em: 585 nm) were then recorded as the mean+/−STDEV from three separate experiments. FIG. 4, Graph A: (1) PBS Buffer alone: 1.7+/−0.32; (2) 1 mM H₂O₂ 812.25+/−89.32; (3) 1 mM glutathione 43.65+/−4.24; (4) 1 mM glutathione disulfide 29.87+/−0.96; (5) 1 mM NaNO₂ 47.10+/−11.06; (6) 1 mM L-cysteine 2.63+/−0.13; (7) 1 mM L-lysine 3.31+/−0.61; (8) 1 mM L-serine 1.70+/−0.13; (9) 1 mM NaCl 40.71+/−1.33; (10) 1 mM NaBr 39.56+/−0.02; (11) 1 mM L-homocysteine 74.32+/−5.27; (12) 100 mM Na₂S 46.97+/−1.47; (13) 1 mM sodium sulfate 38.01+/−2.86; (14) 1 mM sodium thiosulfate 1.72+/−0.22; (15) 1 mM sodium hypochlorite 16.75+/−1.09.

Compound RAH2115 responded well when co-incubated with H₂O₂ as a nearly 500-fold increase in relative fluorescence intensity was observed. However, in the presence of other oxidants (glutathione disulfide, sodium nitrite, and sodium hypochlorite), reductants (glutathione, cysteine, homocysteine, sodium thiosulfate, and hydrogen sulfide), nucleophiles (lysine and serine), and salts (sodium chloride, sodium bromide, and sodium sulfate), the resulting increase in fluorescence intensity was negligible.

The initial response rate of compound RAH2115 was also verified to be directly proportional to the concentration of H₂O₂. Specifically, immediately prior to use, 5 mM and 0.5 mM stock solutions of hydrogen peroxide were prepared in freshly degassed 1× PBS buffer (pH 7.4) using reagent grade H₂O₂ (30%). A 100 μM stock solution of compound RAH2115 was prepared in 200-proof ethanol. 30 μL of a 100 μM stock solution of compound RAH2115 was then added to individual wells on a 96 well plate followed by the addition of H₂O₂ at various concentrations: 1000 μM (60 μL of 5 mM peroxide and 210 μL of buffer), 800 μM (48 μL of 5 mM peroxide and 222 μL of buffer), 600 μM (36 μL of 5 mM peroxide and 234 μL of buffer), 400 μM (24 μL of 5 mM peroxide and 246 μL of buffer), 200 μM (12 μL of 5 mM peroxide and 258 μL of buffer), 100 μM (60 μL of 0.5 mM peroxide and 210 μL of buffer), 75 μM (45 μL of 0.5 mM peroxide and 225 μL of buffer), 50 μM (30 μL of 0.5 mM peroxide and 240 μL of buffer), 25 μM (15 μL of 0.5 mM peroxide and 255 μL of buffer) and 0 μM (270 μL of buffer). Thus, each well contained final concentration of 10 μM for compound RAH2115 and with a total volume of 300 μL. The plate was inserted into a 96-well plate reader (BioTek SYNERGY-HTX hybrid multi-mode reader, Winooski, Vt.) heated to 37° C., and agitated to ensure homogeneity. Fluorescence measurements were recorded and plotted as the change in initial rates of fluorescence intensity vs hydrogen peroxide concentration. This assay was repeated in triplicate and recorded as the mean+/−STDEV from three experiments.

This finding was deemed to be of great significance as the rate of payload release from the scaffold could then be tuned by varying the amount of H₂O₂. In addition, this finding also allowed for reaction-based fluorescent probes based off of this design (i.e., compound RAH2115) to be used to accurately quantify the amount of H₂O₂ in buffered solutions. Indeed, as depicted in FIG. 4, Graph B, when the initial rates of increasing fluorescence intensity of compound RAH2115 were plotted versus hydrogen peroxide concentration, excellent linearity between 0-1 mM was observed (R²=0.99). Moreover, a detection limit for compound RAH2115 was found to be in the low micromolar range. The detection limit of compound RAH2115 was determined to be 1.5 μM and was calculated using following equation: Detection Limit=3σ/κ where σ is the standard deviation of a blank measurement and κ is the resulting slope from FIG. 4, Graph B.

### Example 8

### Imaging Hydrogen Peroxide in Living Cells with Compound RAH2115

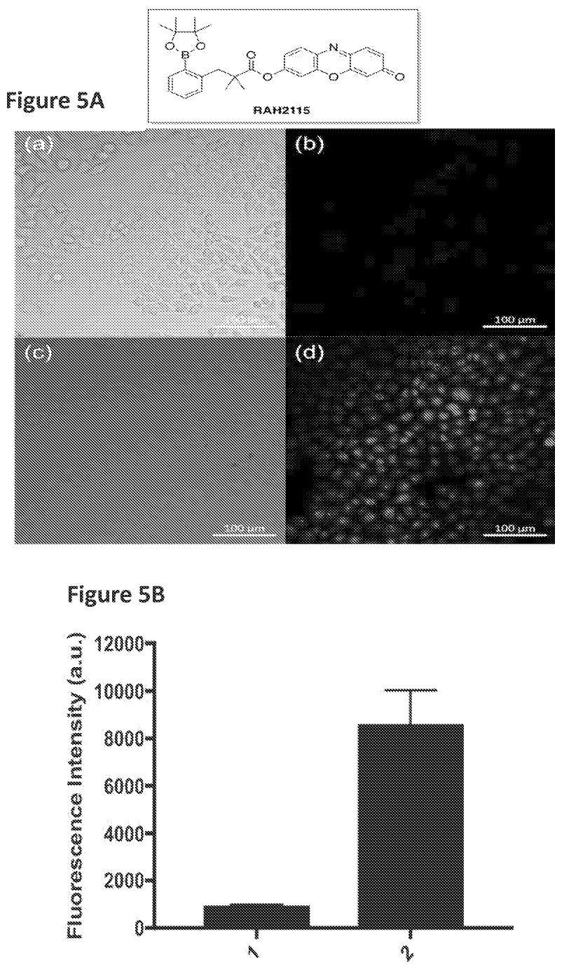

Referring to FIGS. 5A and 5B, to further validate the potential uses of the H₂O₂-sensing framework within a biological context, it was next shown that compound RAH2115 could be used to image hydrogen peroxide within living cells.

HeLa (human cervical cancer) cells were grown and maintained appropriately according to the American Type Culture Collection (Manassas, Va.). Dulbecco's Modified Eagle Medium:F12-K (1:1) was supplemented with 10% fetal bovine serum (FBS), 1% penicillin-streptomycin, and 1% L-glutamine (ThermoFisher). Cells were incubated at 37° C. in a 5% CO₂ atmosphere and split at 70-80% confluency every 2-3 days.

At 70-80% confluency, HeLa cells were seeded on two 35 mm glass-bottom round plates (MatTek Corporation, Ashland, Mass.) at a density of 200 k cells/mL. After adhering overnight, FBS-free media was aspirated and cells were washed with phosphate buffered saline (1×, pH 7.4). Cells in one dish were then incubated with 10 mM Paraquat (Methyl Viologen) for 4 h. Afterwards, both dishes were washed and incubated with compound RAH2115 (10 μM) for 1 hour. After a final PBS wash, both plates were rehydrated in phenol red-free media for imaging. Both control (absence of paraquat) and experimental (presence of paraquat) plates were imaged: ex: 561 nm, em: 587 nm. All cell imaging was acquired using a Carl Zeiss Laser Scanning Confocal Microscope 880 with Airyscan (Oberkochen, Germany) using a 20× objective. Standard incubating conditions were used while imaging (37° C. in a 5% CO₂ atmosphere). Cells were excited using a 561 nm laser at 0.04% power. All parameters within the ZEN software were kept consistent when making direct comparisons (laser power, pinhole, gain, white balance, etc.).

An automated cell selection algorithm within the ImageJ software was used to quantify intracellular fluorescence. Cells from both control and experimental groups were subjected to Gaussian smoothing in order to accurately measure fluorescence within cells based on a minimum threshold against background. This algorithm was applied to three different control and experimental images. Values were exported for plotting and are reported as the average+/−STDEV of three separate images and are consistent with the visual inspection of FIG. 5A.

Under these conditions, no fluorescent cells were observed (See FIG. 5A, panels (a) and (b), and FIG. 5B). However, given the detection limit of compound RAH2115 and the natural abundance of peroxide within most cells (<1 μm; see, e.g., J. R. Stone, S. Yang, Antioxid. Redox Signal. 2006, 8, 243-270), this finding was not unexpected and instead further signifies the stability of our framework, even within a complex biological setting. Conversely, when HeLa cells were first pre-treated with paraquat, a known H₂O₂ stimulator (see, e.g., B. C. Dickinson, C. J. Chang, J. Am. Chem. Soc. 2008, 130, 9638-9639 and A. L. McCormack, M. Thiruchelvam, A. B. Manning-Bog C. Thiffault, J. W. Langston, D. A. Cory-Slechta, D. A. Di Monte, Neurobiol. Dis. 2002, 10, 119-127), the addition of compound RAH2115 resulted in a strong red fluorescence within cells, further accentuating the high selectivity of the scaffold and its ability to release a payload of interest under conditions of oxidative stress (See FIG. 5A, panels (c) and (d), and FIG. 5B).

### Example 9

### Synthesis Persulfide Donor Scaffolds Compounds 4a and 4b

Given these results, it was proposed to convert this novel H₂O₂-sensing scaffold into a reactive oxygen species (ROS)-responsive persulfide donor. In particular, the initial cellular studies suggested that such a donor would likely afford the preferential release of persulfides within biological systems with elevated levels of ROS, thereby providing a useful chemical tool for probing the therapeutic potential of RSS and their ability to combat oxidative stress-related diseases.

The compound 4a was first generated as a convenient way to monitor persulfide release from the scaffold.

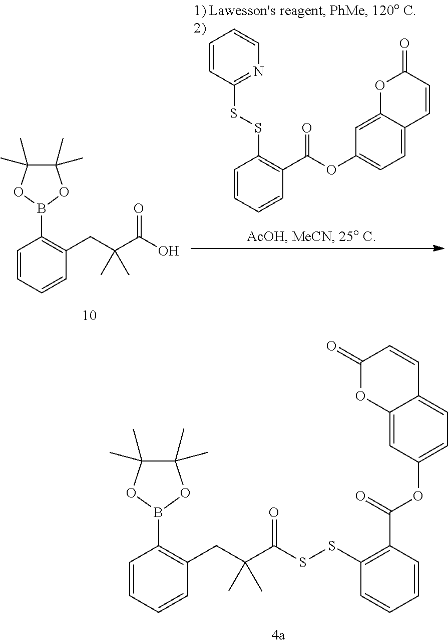 [SMILES: CC(C)(Cc1ccccc1B1OC(C)(C)C(C)(C)O1)C(=O)O ; O=C(Oc1ccc2ccc(=O)oc2c1)c1ccccc1SSc1ccccn1 ; CC(C)(Cc1ccccc1B1OC(C)(C)C(C)(C)O1)C(=O)SSc1ccccc1C(=O)Oc1ccc2ccc(=O)oc2c1 ; "4a"]

Specifically, to a solution of carboxylic acid compound 10 (55 mg, 0.18 mmol) in toluene (5 mL) was added Lawesson's reagent (22 mg, 0.054 mmol). The mixture was heated to 120° C. in a thick-walled pressure vessel and allowed to stir at elevated temperature overnight. The solution was then cooled, and solvent was removed by rotary evaporation. The resulting mixture was dissolved in dichloromethane, washed with 0.5 M HCl (aq) and brine, and concentrated under reduced pressure. A portion of the crude thioacid (47 mg, 0.14 mmol), activated disulfide (Liu, C.; Pan, J.; Li, S.; Zhao, Y.; Wu, L. Y.; Berkman, C. E.; Whorton, A. R.; Xan, M. Capture and Visualization of Hydrogen Sulfide by a Fluorescent Probe. Angew. Chemie Int. Ed. 2011, 50 (44), 10327-10329; 59 mg, 0.14 mmol), and acetic acid (412 μL, 0.725 mmol) were then mixed in acetonitrile (2 mL) and allowed to stir overnight at ambient temperature. After consumption of the thioacid, determined by TLC, the reaction mixture was then diluted with dichloromethane, washed with deionized water, and concentrated under reduced pressure. The resultant extract was purified via column chromatography (gradient of 100% hexanes to 100% ethyl acetate) to yield compound 4a as a yellow solid (44 mg, 50%). ¹H NMR (400 MHz, CDCI₃) δ=8.26 (dd, J=7.8, 1.1 Hz, 1H), 7.85 (dd, J=7.4, 1.6 Hz, 1H), 7.73 (d, J=9.6 Hz, 1H), 7.63-7.51 (m, 3H), 7.38-7.28 (m, 3H), 7.29-7.20 (m, 2H), 7.11 (dd, J=7.5, 1.3 Hz, 1H), 6.43 (d, J=9.6 Hz, 1H), 3.44 (s, 2H), 1.36 (s, 12H), 1.31 (s, 6H); ¹³C NMR (101 MHz, CDCl₃) δ=202.26, 164.16, 160.33, 154.79, 153.16, 143.23, 142.86, 141.74, 136.29, 133.81, 131.94, 130.93, 130.61, 128.66, 126.37, 126.23, 125.97, 125.70, 118.67, 116.92, 116.27, 110.70, 83.72, 51.87, 43.20, 24.99, 24.52; ESI/MS calculated for [C₃₃H₃₃BNaO₇S₂⁺] (M+Na⁺) requires m/z=639.17, found 639.19.

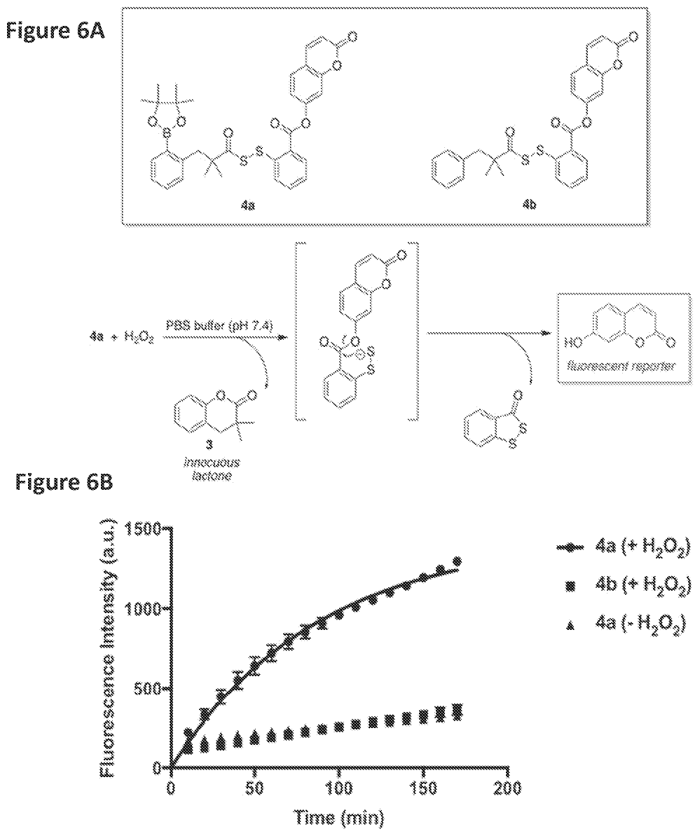

As illustrated in FIG. 6A, it was hypothesized that upon reacting with peroxides, the resulting phenol would once again undergo rapid lactonization resulting in persulfide release. Once liberated, the free persulfide would then undergo a second cyclization onto the proximal ester with the concurrent release of 7-hydroxycoumarin. Thus, the resulting increase in fluorescence intensity at the characteristic wavelength of 7-hydroxycoumarin could be monitored and would be directly proportional to the amount of released persulfide.

As a control, compound 4b was generated. Compound 4b lacks the boronate ester, thereby rendering this proposed mechanistic pathway nonoperational.

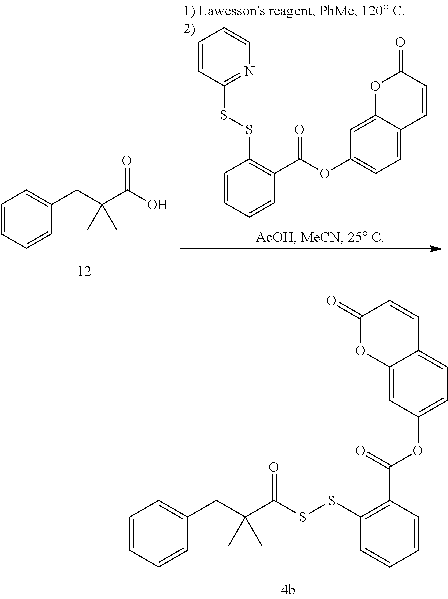 [SMILES: CC(C)(Cc1ccccc1)C(=O)O ; O=C(Oc1ccc2ccc(=O)oc2c1)c1ccccc1SSc1ccccn1 ; CC(C)(Cc1ccccc1)C(=O)SSc1ccccc1C(=O)Oc1ccc2ccc(=O)oc2c1]

To prepare compound 4b, to a solution of carboxylic acid compound 12 (0.150 g, 0.84 mmol) in toluene (10 mL) was added Lawesson's reagent (84 mg, 0.21 mmol). The mixture was heated to 120° C. in a thick-walled pressure vessel and allowed to stir at elevated temperature overnight. The solution was then cooled, and solvent was removed by rotary evaporation. The resulting mixture was dissolved in dichloromethane, washed with 0.5 M HCl(aq) and brine, and concentrated under reduced pressure. A portion of the crude thioacid (40 mg, 0.21 mmol), activated disulfide¹ (85 mg, 0.21 mmol), and acetic acid (59 μL, 1.03 mmol) were then mixed in acetonitrile (2 mL) and allowed to stir overnight. The crude mixture was then diluted in dichloromethane, washed with deionized water, and concentrated under reduced pressure. The resultant extract was purified via column chromatography (gradient of 100% hexanes to 100% ethyl acetate) to yield compound 4b as a pale yellow solid (47 mg, 47%). ¹H NMR (400 MHz, CDCI₃) δ=8.25 (dd, J=7.8, 1.5 Hz, 1H), 7.73 (d, J=9.5 Hz, 1H), 7.60-7.42 (m, 3H), 7.40-7.20 (m, 6H), 7.18-7.08 (m, 2H), 6.42 (d, J=9.6 Hz, 1H), 2.99 (s, 2H), 1.38 (s, 6H); ¹³C NMR (101 MHz, CDCl₃) δ=201.79, 164.16, 160.32, 154.78, 153.13, 142.87, 141.43, 136.72, 133.84, 131.92, 130.51, 128.68, 128.18, 126.80, 126.44, 126.26, 125.80, 118.64, 116.94, 116.27, 110.68, 51.68, 46.31, 25.15.

### Example 10

### Fluorescence Experiments with Compounds 4a and 4b

Referring to FIGS. 6A and 6B, compounds 4a and 4b (20 μM) were exposed to H₂O₂ (200 μM) in PBS buffer (pH 7.4) at 37° C. and the resulting fluorescence intensity was monitored over a period of 3 hours. Immediately prior to use, a 200 μM stock solutions of compound 4a and compound 4b were prepared in cell-grade dimethylsulfoxide (DMSO), and a 200 μM stock of cetyltrimethylammonium bromide (CTAB) was prepared in PBS buffer (pH 7.4). Next, a 2 mM hydrogen peroxide stock solution was prepared by diluting 30% hydrogen peroxide in freshly degassed PBS Buffer (pH 7.4). 30 μL of compound 4a (or compound 4b) was then added to a 96 well plate and diluted with 90 μL of PBS buffer and 150 μL of the CTAB stock solution. The assay was then initiated by the addition of 30 μL, of the 2 mM hydrogen peroxide stock solution rendering a final concentration of 20 μM for compound 4a (or compound 4b), 200 μM of hydrogen peroxide, and 100 μM of CTAB. The control reactions without peroxide were conducted by adding 30 μL of the 200 μM stock of compound 4a to a 96 well plate and diluting with 120 μL of PBS buffer and 150 μL of 200 μM CTAB to make a final concentration of 20 μM of compound 4a and 100 μM of CTAB. The plate was inserted into a 96-well plate reader (BioTek SYNERGY-HTX multi-mode reader, Winooski, Vt.) heated to 37° C., and agitated to ensure homogeneity. The plate was read continuously using a 360/40 nm fluorescence filter and values were recorded as the mean+/−STDEV from three experiments. Under these conditions, the reaction between compound 4a and H₂O₂ displayed pseudo first-order kinetics with a half-life of 60.5 min and a k_{obs} of 0.012 min⁻¹ (FIG. 6B).

While the relative fluorescence intensity was found to significantly increase over time with compound 4a, when compound 4b was used, the subsequent increase in fluorescence was found to be trivial. Moreover, when compound 4a was incubated in PBS buffer alone, it was found to display a nearly identical fluorescence profile to that of compound 4b indicating that the slight increase in fluorescence over time is likely attributable to the direct hydrolysis of the more labile coumarin ester. Under these conditions, the reaction between compound 4a and H₂O₂ was found to have a half-life of 60.5 min and with a pseudo first-order rate constant of 0.012 min⁻¹, values which are comparable to those of other previously reported ROS-activated RSS donors.

### Example 11

### Synthesis of Compound RAH393 and Control Compound 5

After further confirming the stability of the novel scaffold, as well as its ability to selectively release persulfides in response to H₂O₂, persulfide donor compound RAH393 was synthesized (see FIG. 18 and the Scheme below).

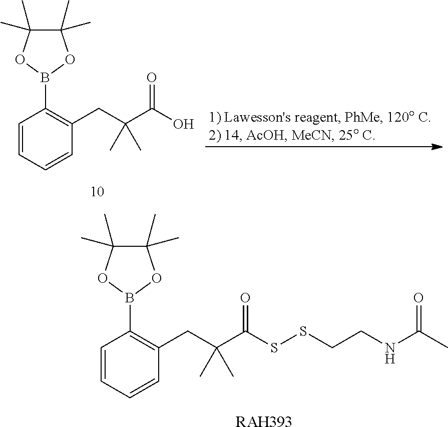 [SMILES: CC(C)(Cc1ccccc1B1OC(C)(C)C(C)(C)O1)C(=O)O ; CC(=O)NCCSSC(=O)C(C)(C)Cc1ccccc1B1OC(C)(C)C(C)(C)O1 ; "RAH393"]

Specifically, to a solution of carboxylic acid compound 10 (81 mg, 0.27 mmol) in toluene (10 mL) was added Lawesson's reagent (32 mg, 0.080 mmol). The mixture was heated to 120° C. in a thick-walled pressure vessel and allowed to stir at elevated temperature overnight. The solution was cooled, solvent was removed by rotary evaporation, and the resulting mixture was dissolved in dichloromethane and washed with 0.5M HCl(aq) and brine. The crude thioacid product was then concentrated under reduced pressure, combined with activated disulfide compound 14 (0.122 g, 0.53 mmol), acetic acid (152 μL, 2.66 mmol), diluted with acetonitrile (2 mL), and allowed to stir overnight at ambient temperature under an atmosphere of nitrogen. The crude mixture was then diluted with dichloroethane, washed with deionized water, and concentrated under reduced pressure. The resultant extract was purified via column chromatography (gradient of 100% hexanes to 100% ethyl acetate) to yield compound RAH393 as a pale yellow solid (23 mg, 20%). ¹H NMR (400 MHz, CDCI₃) δ=7.74 (dd, J=7.4, 1.6 Hz, 1H), 7.26 (td, J=7.5, 1.6 Hz, 1H), 7.16 (td, J=7.4, 1.3 Hz, 1H), 7.02 (dd, J=7.7, 1.2 Hz, 1H), 6.72 (s, 1H), 3.30 (s, 2H), 3.24 (q, J=5.8 Hz, 2H), 2.75-2.65 (m, 2H), 2.00 (s, 3H), 1.29 (s, 12H), 1.18 (s, 6H); ¹³C NMR (101 MHz, CDCl₃)δ=207.12, 170.32, 143.09, 136.20, 130.66, 130.54, 126.01, 83.72, 51.83, 43.47, 39.10, 36.68, 24.98, 24.73, 23.46; ESI/MS calculated for [C₂₁H₃₃BNO₄S₂⁺] (M+H⁺) requires m/z=438.19, found 438.18.

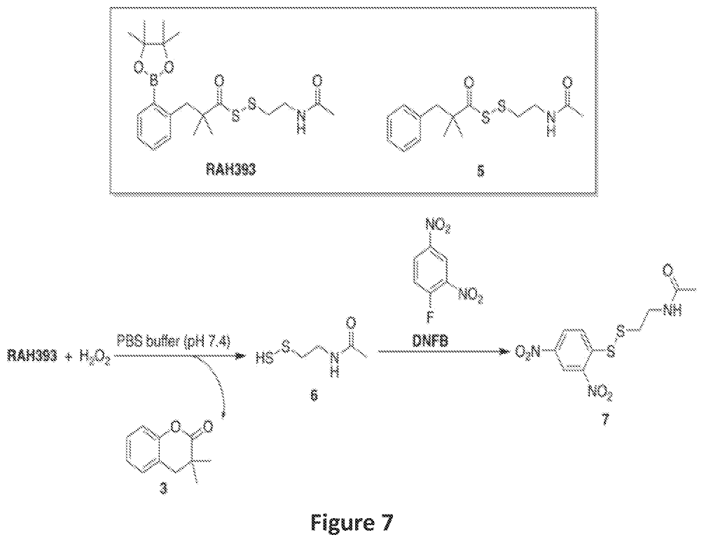

N-acetylcysteamine was chosen due to its reported high biocompatibility and strong antioxidant effects which would likely render its persulfide as a promising combatant of ROS. See, e.g., F. Shams, D. Oladiwura, K. Ramaesh, I. Livingstone, Clin. Ophthalmol. 2014, 2077-2084. However, prior to evaluating the therapeutic potential of compound RAH393, the ability of the scaffold to release this specific persulfide in response to H₂O₂ was first confirmed. Referring to FIG. 7, compound RAH393 was incubated along with 2,4-dinitrofluorobenzene (DNFB) in an effort to trap the ensuing persulfide as a stable disulfide.

For comparison, control compound 5, which lacks the requisite H₂O₂-responsive moiety, was synthesized.

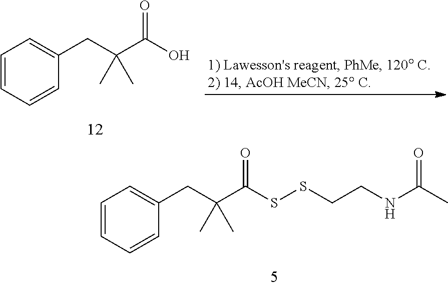 [SMILES: CC(C)(Cc1ccccc1)C(=O)O ; CC(=O)NCCSSC(=O)C(C)(C)Cc1ccccc1]

Specifically, to a solution of carboxylic acid compound 12 (0.100 g, 0.56 mmol) in toluene (10 mL) was added Lawesson's reagent (0.113 g, 0.28 mmol). The mixture was heated to 120° C. in a thick-walled pressure vessel and allowed to stir at elevated temperature overnight. The solution was cooled, and solvent was removed by rotary evaporation. The crude mixture was dissolved in dichloromethane, washed with 0.5 M HCl(aq) and brine, and concentrated under reduced pressure. A portion of the crude thioacid product (90 mg, 0.464 mg) was combined with activated disulfide compound 14 (0.106 g, 0.46 mmol), acetic acid (132 μL, 2.32 mmol) and allowed to stir overnight. The crude mixture was then diluted with dichloromethane, washed with deionized water, and concentrated under reduced pressure. The resultant extract was purified via column chromatography (gradient of 100% hexanes to 100% ethyl acetate) to yield compound 5 as a yellow oil (37 mg, 26%). ¹H NMR (400 MHz, CDCI₃) δ=7.28-7.22 (m, 3H), 7.10 (dd, J=7.8, 1.8 Hz, 2H), 6.74 (s, 3.31 (q, J=5.8 Hz, 2H), 2.94 (s, 2H), 2.84-2.69 (m, 2H), 2.07 (s, 3H), 1.30 (s, 6H); ¹³C NMR (101 MHz, CDCl₃) δ=206.51, 170.32, 136.57, 130.34, 128.17, 126.88, 51.60, 46.38, 39.05, 36.74, 25.12, 23.45; ESI/MS calculated for [C₁₅H₂₂NO₂S₂⁺] (M+H⁺) requires m/z=312.11, found 312.08.

The synthetic procedure for preparing active disulfide compound 14 is described below.

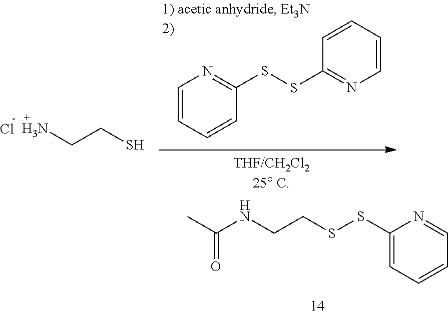

To a dry round-bottom flask was added cysteamine hydrochloride (1.00 g, 4.54 mmol) in dry tetrahydrofuran (4 mL) and the resulting solution was then diluted with anhydrous dichloromethane (50 mL). Anhydrous triethylamine (1.27 mL, 9.08 mmol) was then added to the flask, followed by the dropwise addition of acetic anhydride (429 μL, 4.54 mmol) and the mixture was allowed to stir under an atmosphere of nitrogen overnight at ambient temperature. Dipyridyl disulfide (1.00 g, 4.54 mmol) was then added and the reaction was allowed to stir at ambient temperature for 24 h. The mixture was then washed with deionized water and NaHCO₃(aq). The resultant organic solution was dried over anhydrous sodium sulfate, concentrated under reduced pressure, and purified by silica gel column chromatography (90:10 mixture of ethyl acetate:methanol) to give compound 14 as a yellow solid (0.53 g, 51%). ¹H NMR (400 MHz, CDCl₃) δ=8.50 (ddd, J=4.9, 1.8, 1.0 Hz, 1H), 7.63 (ddd, J=8.1, 7.3, 1.8 Hz, 1H), 7.53 (dt, J=8.1, 1.0 Hz, 1H), 7.15 (ddd, J=7.3, 4.9, 1.1 Hz, 1H), 3.55 (q, J=6.0 Hz, 2H), 2.98-2.88 (m, 2H), 2.02 (s, 3H); ¹³C NMR (101 MHz, CDCl₃) δ=170.14, 159.15, 149.75, 137.02, 121.34, 121.13, 38.88, 37.36, 23.40; ESI/MS calculated for [C₉H₁₃N₂OS₂⁺] (M+H⁺) requires m/z=229.05, found 228.96.

### Example 12

### Validation of Compound RAH393 as an ROS-Activated Persulfide Donor

Initially, 5 mM stock solutions of compound RAH393, compound 5, and 2,4-dinitrofluorobenzene (DNFB) were prepared in 200-proof ethanol. In addition, a 100 mM stock solution of hydrogen peroxide in ammonium bicarbonate buffer (1 mM, pH 7.0) was prepared immediately prior to use. To a conical vial containing 1500 μL of 1M ammonium bicarbonate was then added 80 μL of the 5 mM stock of compound RAH393 (or control compound 5), 400 μL of the 5 mM DNFB stock, and 20 μL of 100 mM hydrogen peroxide giving a final concentrations of 200 μM compound RAH393 (or compound 5), 1 mM DNFB, and 1 mM H₂O₂. At the designated timepoints, a 100 μL aliquot of the room-temperature reaction was diluted with 900 μL of HPLC-grade methanol with 0.1% formic acid for analysis. 75 μL of the resultant solution was then injected onto C18 reverse phase column (Agilent Technologies, SB-C18 Analytical HPLC Col. 4.6×150) and eluted with HPLC-grade methanol (0.1% formic acid) and HPLC-grade water (0.1% formic acid) at a flow rate of 200 μL/min using the following gradient: 0-2 min, 90:10 water:methanol; 2-8 min, 40:60 water:methanol; 8-30 min, 10:90 water:methanol; 30-40 min, 5:95 water:methanol. Retention times of lactone compound 3 (25.0 min), compound RAH393 (35.6 min), compound 5 (27.7 min), and DNFB (16.5 min) were independently validated. When this assay was run using compound RAH393, in addition to compound 7 (21.7 min), the tri- and tetrasulfides of N-acetylcysteamine were also present (14.1 min and 16.2 min, respectively), further confirming persulfide release from compound RAH393.

It was found that incubating compound RAH393 (200 mM) with H₂O₂ (1 mM) and DNFB (1 mM), persulfide release indeed occurs as formation of disulfide compound 7 was confirmed by LCMS. In addition, persulfide release from our scaffold was further verified by the linear increase in the production of lactone compound 3, again verified by LCMS, over a period of 25 min. The intermediate phenol that precedes lactone formation was not able to be identified via LCMS, thus further verifying that once the scaffold reacts with H₂O₂, the subsequent cyclization event is rapid. These observations, along with the fact that compound 5 proved unsuccessful in yielding compound 7 under identical conditions, further corroborates that persulfide release does indeed occur from the scaffold, in response to peroxides, and via the hypothesized mechanistic pathway as opposed to nonspecific hydrolysis.

### Example 13

### Evaluation of Compound RAH393 in a Biological Setting

First, compound RAH393 and lactone compound 3 were tested for cytotoxicity in HeLa cells. A population of cells (>6×10⁶ cells/mL as determined with a hemocytometer) was diluted with FBS-free DMEM:F12-K, 1:1, 1% penicillin-streptomycin, and 1% L-glutamine to give a final concentration of 50,000 cells/mL. To a 96-well plate was added 100 μL of cell media solution resulting in a total of 5 k cells per well. Plates were then incubated at 37° C. in an atmosphere of 5% CO₂ and 95% humidified air for 24 h. Fresh culture media (100 μL) was then added to each well of cells resulting in 200 μL of media per well followed by the addition of compound RAH393 or compound 3 at various concentrations (20 mM stock in biological-grade DMSO, resulting in no more than 1% DMSO per well). After incubating for 5 h, a phosphatase assay was used to establish cell viability as follows: Media was removed from each well and replaced with 100 μL of phosphatase solution (100 mg of phosphatase substrate, in 30 mL of 0.1 M NaOAc, pH 5.5, 0.1% Triton X-100 buffer). Following an incubation period of 30 min, 50 μL of 0.10 M NaOH was added to each well and viability was assessed spectrophotometrically at 405 nm using a 96-well plate reader (BioTek SYNERGY-HTX multi-mode reader, Winooski, Vt.). Assays were performed in triplicate and presented as the mean+/−STDEV. It was determined that neither compound RAH393 nor lactone compound 3 were toxic toward HeLa cells.

Hydrogen peroxide was also tested for cytotoxicity in HeLa cells. A population of cells (>6×10⁶ cells/mL as determined with a hemocytometer) was diluted with FBS-free DMEM:F12-K, 1:1, 1% penicillin-streptomycin, and 1% L-glutamine to give a final concentration of 50,000 cells/mL. To a 96-well plate was added 100 μL of cell media solution resulting in a total of 5 k cells per well. Hydrogen peroxide was then added to each well at varying concentrations (50-400 μM) and cultures were then incubated for 1 h. Viability was assessed using Cell Counting Kit-8 (Dojindo Molecular Technologies, Rockville, Md.) and the assay was performed in triplicate and presented as the mean+/−STDEV.

Next, the protective effects of compound RAH393 on cultured HeLa cells which were under oxidative stress were examined. A population of cells (>6×10⁶ cells/mL as determined with a hemocytometer) was diluted with FBS-free DMEM:F12-K, 1:1, 1% penicillin-streptomycin, and 1% L-glutamine to give a final concentration of 50,000 cells/mL. To a 96-well plate was added 100 μL of cell media solution resulting in a total of 5 k cells per well. Plates were then incubated at 37° C. in an atmosphere of 5% CO₂ and 95% humidified air for 24 h. Confluent cells were then pre-incubated in FBS-free media with compound RAH393 (or compound 5) (200 μM) for 1 h followed by treatment with H₂O₂ (200 μM) for an additional 1 h incubation period. Control wells with H₂O₂ alone (200 μM) were also evaluated on the same plate. After a total incubation period of 2 h, cell contents were then replaced with 100 μL of fresh FBS-free media with 10% CCK-8. Cells were then incubated for 3 h at 37° C. and cell viability was assessed by measuring the resulting absorbance at 450 nm using a 96-well plate reader (BioTek SYNERGY-HTX multi-mode reader, Winooski, Vt.).

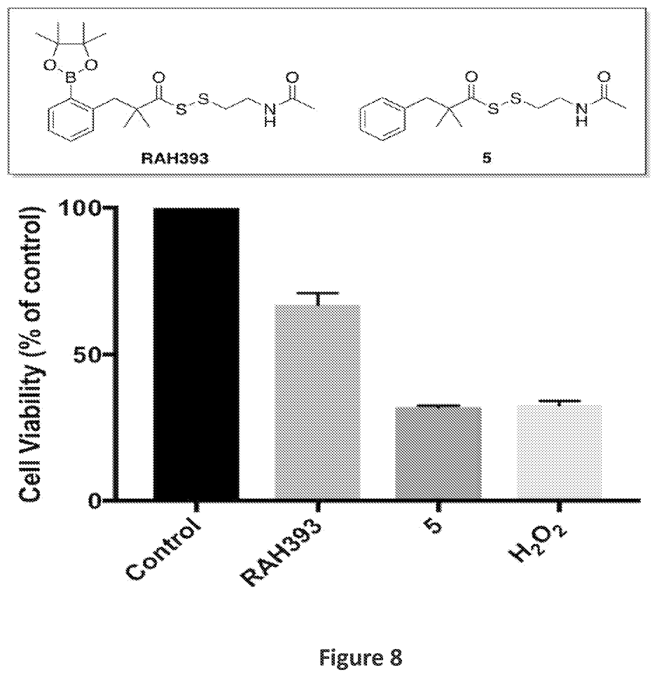

Referring to FIG. 8, incubating HeLa cells with 200 μM H₂O₂ over a period of 1 hour reduced cell viability to just 33%. However, when HeLa cells were first pre-treated with compound RAH393 (200 μM) prior to the 1-hour incubation period with H₂O₂ (200 μM), an astounding 100% increase in cell viability was observed (See FIG. 8). As a control, compound 5 was again tested alongside compound RAH393 and, under identical conditions, was found to provide no protection against H₂O₂-induced oxidative stress. Therefore, the results from this study clearly highlight the protective effects of compound RAH393 and its potential as a combatant of oxidative stress-related diseases.

The foregoing is illustrative of the present invention and is not to be construed as limiting thereof. The invention is defined by the following claims, with equivalents of the claims to be included therein.

1. A compound of Formula I: 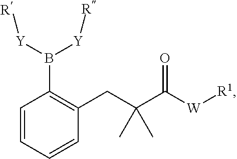
wherein:
R¹ is H, halo, alkyl, heteroalkyl, aryl, or heteroaryl, wherein the alkyl, heteroalkyl, aryl, or heteroaryl is optionally substituted, and/or R¹ comprises at least one of a prodrug moiety and a reporter moiety;
each Y is independently NRₐ or O;
W is NR_{b}, S, or O;
Rₐ and R_{b} are each independently H or alkyl, optionally substituted; and
R′ and R″ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, —OH, alkoxy, carbonyl and/or carboxy groups;
or a pharmaceutically acceptable salt thereof.

2. The compound of claim 1, wherein said compound of Formula I is a compound of Formula Ia: 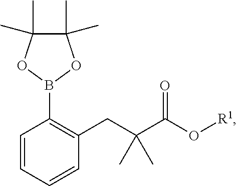
wherein:
R¹ is as defined above;
or a pharmaceutically acceptable salt thereof.

3. The compound of claim 1, wherein R¹ comprises a reporter moiety.

4. The compound of claim 3, wherein the reporter moiety produces a fluorescent compound when the compound reacts with a reactive oxygen species.

5. The compound of claim 4, wherein the reporter moiety is:

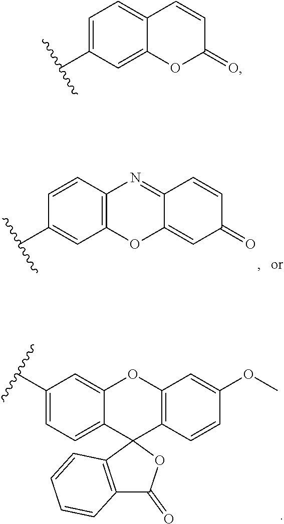 [SMILES: *c1ccc2ccc(=O)oc2c1 ; *c1ccc2nc3ccc(=O)cc-3oc2c1 ; *c1ccc2c(c1)Oc1cc(OC)ccc1C21OC(=O)c2ccccc21]

6. The compound of claim 1, wherein R¹ comprises a prodrug moiety that produces an active drug when the compound reacts with a reactive oxygen species.

7. The compound of claim 6, wherein the active drug is a phenolic drug.

8. A method of detecting the presence of hydrogen peroxide in a cell and/or tissue comprising contacting the cell and/or tissue with a compound of claim 3 and detecting the presence of a lactone and/or a fluorescent compound, wherein the presence of a lactone and/or a fluorescent compound indicates the presence of the hydrogen peroxide in the cell and/or tissue.

9. The method of claim 8, wherein the concentration of the lactone and/or fluorescent compound is used to determine the concentration of the hydrogen peroxide in the cell and/or tissue.

10. The method of claim 8, wherein the contacting is carried out in vitro.

11. The method of claim 8, wherein the contacting is carried out in vivo.

12. A compound of Formula II: 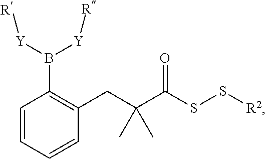
wherein:
R² is H, alkyl, heteroalkyl, aryl, or heteroaryl, wherein the alkyl, heteroalkyl, aryl, or heteroaryl is optionally substituted, and/or wherein R² comprises at least one of a reporter moiety and a prodrug moiety,
each Y is independently NR_{c} or O;
R_{c} is H or alkyl, optionally substituted; and
R′ and R″ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, —OH, alkoxy, carbonyl and/or carboxy groups; and
or a pharmaceutically acceptable salt thereof.

13. The compound of claim 12, wherein said compound is a compound of Formula IIa: 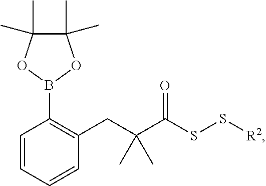
wherein R² is as defined above, or a pharmaceutically acceptable salt thereof.

14. The compound of claim 12, wherein R² is —(CH₂)₂NHC(═O)CH₃.

15. The compound of claim 12, wherein R² is: 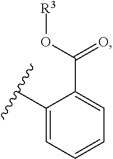
wherein R³ is the reporter moiety.

16. The compound of claim 15, wherein the reporter moiety produces a fluorescent compound when the compound reacts with a reactive oxygen species.

17. The compound of claim 15, wherein the reporter moiety is:

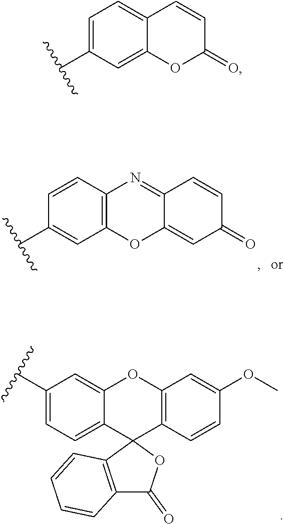 [SMILES: *c1ccc2ccc(=O)oc2c1 ; *c1ccc2nc3ccc(=O)cc-3oc2c1 ; *c1ccc2c(c1)Oc1cc(OC)ccc1C21OC(=O)c2ccccc21]

18. A method of producing a persulfide in the presence of hydrogen peroxide in a cell and/or tissue, comprising contacting the cell and/or tissue with a compound of claim 12, whereby the persulfide is produced when the hydrogen peroxide reacts with the compound.

19. The method of claim 18, wherein the compound comprises a reporter moiety and the method further comprises detecting the reporter moiety.

20. The method of claim 18, wherein the contacting is carried out in vitro.

21. The method of claim 18, wherein the contacting is carried out in vivo.
